(19) United States
(12) Patent Application Publication
(10) Pub. No.: US 2025/0209490 A1
(43) Pub. Date: Jun. 26, 2025

(54) Support Garment with Feedback and Incentive System

(71) Applicant: Suzan SZOLLAR, Menlo Park, CA (US)

(72) Inventor: Suzan SZOLLAR, Menlo Park, CA (US)

(21) Appl. No.: 19/080,909

(22) Filed: Mar. 16, 2025

Related U.S. Application Data
(63) Continuation-in-part of application No. PCT/US2023/074309, filed on Sep. 15, 2023.
(60) Provisional application No. 63/373,054, filed on Aug. 21, 2022.

(51) Int. Cl. G06Q 30/0226; A61B 5/00; A61F 5/02; G06Q 30/0601; G16H 40/67

(52) U.S. Cl. CPC G06Q 30/0233; A61B 5/486; A61F 5/026; G06Q 30/0641; G16H 40/67

(57) ABSTRACT

The disclosure provides a posture support garment device with a back band, axilla region portions extending under the armpits and over shoulders, scapular region portions forming a cross-back structure, underarm regions, and a forward fastening portion. This design applies tension to improve posture while maintaining position during movement. An incentive system is disclosed with usage sensors, data analysis, value establishment, and credit exchange mechanisms. A flexible framework allows expanding motivations and contributions. A feedback system incorporates sensors to capture user signals, haptic devices for sensory feedback, and functional elements to analyze data and provide feedback, assisting users in regulating physiological states. The garment's unique structure, combined with integrated sensing and feedback capabilities, offers a comprehensive solution for posture improvement and user engagement.

### CROSS-REFERENCE TO RELATED APPLICATIONS

This application is a continuation-in-part bypass application of International Application No. PCT/US2023/074309, filed Sep. 15, 2023, entitled “Support Garment with Feedback and Incentive System,” which claims priority to U.S. Provisional Patent Application No. 16/376,054, titled “Support Garment with Feedback and Incentive System,” filed Sep. 16, 2022, the contents of each are hereby incorporated by reference in their entireties.

### FIELD OF INVENTION

The present disclosure relates to posture support garments, and more particularly to a posture support garment device with integrated sensors and haptic feedback, coupled with an incentive system for encouraging consistent use and promoting long-term posture improvement.

### BACKGROUND

Poor posture has been linked to various physical and mental health issues, including numbness and tingling in hands, arms and shoulders, as well as stress on ligaments and joints. Studies estimate that in the U.S. alone, $635 billion is spent annually on medical bills and lost productivity related to back pain, with 264 million lost workdays per year. Back and neck pain are among the top five causes of disability globally.

Prolonged sitting, especially in front of computers, has been associated with poor health outcomes since as early as the 1840s when it was first observed in bus drivers. Modern estimates suggest that office workers spend an average of ten or more hours daily in front of computers. Additionally, recent studies have found that users spend four or more hours a day looking down at their phones, either sitting or standing, which strains the neck and cervical spine.

Sitting or standing in a slouched position, with shoulders rounded forward, misaligns bones and joints, creating abnormal wear and putting stress on ligaments. This can lead to short and long-term back and neck pain, muscle strain, overuse disorders, and reduced lung capacity, which may result in fatigue, stress, and respiratory problems. Disorders resulting from prolonged subjection of the body to these positions include conditions colloquially known as “Gameboy Back,” “Tech Neck,” and “Text Neck.”

Prior posture improvement solutions encompass a wide range of approaches, from physically restraining devices and proprioceptive garments worn on the body to furniture such as ergonomic chairs and standing desks. Software solutions and wearable electronics with sensors for tracking body position have also been developed. However, many of these solutions have limitations or drawbacks.

U.S. Patent Publication No. 20050197607A1 to Brown discloses a posture improvement device comprising a form-fitting garment that envelops the patient's torso and upper arms. The device includes diagonal tension straps secured to the back portion with free ends attachable to the chest and waist portions. While this device aims to improve posture by applying pressure and torque to train muscles through neuromuscular stimulation, it may be uncomfortable for extended wear and limit range of motion.

U.S. Patent Application Publication No. U.S. Pat. No. 20,190,282821A1 to Zoll describes a wearable medical device for monitoring a patient's cardiac condition. The device includes ECG electrodes for continuous skin contact and touch electrodes for intermittent contact. While this device focuses on cardiac monitoring rather than posture correction, it demonstrates the potential for integrating sensors and feedback mechanisms into wearable garments.

Other prior art, such as U.S. Pat. No. 8,556,840, discloses posture correction braces with tightening mechanisms that allow users to adjust shoulder straps to pull the shoulders back. However, these devices often rely on rigid components that can cause discomfort and may lead to muscle atrophy over time. Additionally, many existing solutions are either worn over clothes, limiting when and where they can be used, or are bulky and uncomfortable when worn under clothing.

Restraining devices such as braces to improve posture that resemble backpack straps have been disclosed in various patents and patent applications. For example, U.S. Pat. No. 8,308,670 to Sandifer et al., U.S. Pat. No. 8,556,840 to Burke et al., U.S. Patent No. 8,905,956 to Waeger, U.S. Pat. No. 9,456,919 to Pollack, U.S. Patent No. 9,504,280 to Levian, U.S. Patent No. 9,572,705 to Ingimundarson et al., U.S. Pat. No. 9,931,236 to Williamson et al., U.S. Pat. No. 10,406,014 to Collier et al., and U.S. Patent Application Publication No. 2014/0221893 to Modglin, describe devices with a plurality of straps extending over the collarbone, joined to a piece centered along the spine, and additional components to secure the device to the body such as a waist belt. These devices may be typically made of multiple pieces of rigid materials and have been associated with muscle atrophy and weakness over time, potentially leading to long-term negative health consequences. In some cases, these devices may be worn over clothes, which may limit when and where they can be worn. In other instances, when worn under clothing, these devices may be bulky and may rub against the skin due to the lack of elasticity and softness, potentially causing abrasion and injury to the skin.

Stretchy devices worn over clothes may be easily displaced during modest movements such as arm extension or vigorous activities like exercise, potentially limiting their range of usability and effectiveness. For instance, U.S. Pat. No. 8,808,212 to Redmond describes a shoulder support device comprising an elastic continuous ring band that can be tightened by twisting at the center and worn over the shoulders. While this design may pull the shoulders back, it may provide limited stability during a range of motion. The twist in the center may create bulk in the middle of the back, potentially causing discomfort.

Devices that incorporate a protrusion in the center of the back along the spine to fix the restraint in place may make it uncomfortable for users to lean back. Furthermore, wearing a device over clothes may require users to select clothing that accommodates the aid, potentially limiting how often the device is worn and the types of clothes the user can wear. This visibility may also restrict usage in certain social or professional situations where users may not feel comfortable wearing a visible device.

Therapeutic undergarments, such as bras or bra-like undergarments intended to provide somatic or proprioceptive correction, may rely on straps that extend over clavicle bones, originally intended to support the breasts. For example, U.S. Pat. No. 10,721,975 to Liu, U.S. Pat. No. 8,047,893 to Fenske, and U.S. Patent Application Publication No. 2021/0186125 to Lee describe such designs. The position of these straps over the collarbone may hinder their effectiveness in regulating healthy posture alignment. In cases where tension is required, these designs may rely on attachment to the back of the band that runs around the under-breast torso region. This band may need to be worn tight to prevent the back portion of the strap from being pulled up by the force of the shoulders.

Similarly, garments resembling shirts designed with straps or bands extending over the collarbone, either attached or incorporated into the material, may manifest comparable limitations. Examples include U.S. Pat. Nos. 7,871,388 to 9,168,167 to Brown, U.S. Pat. No. 9,009,863 to Decker, U.S. Pat. No. 9,226,845 to Troncoso, and U.S. Pat. No. 9,883,703 to Schultz. These shirt-like garments are often tight-fitting to maintain their position and perform their intended function. Such tight garment structures may lead to discomfort, especially for women with varying breast sizes. The shirts, which may offer compression in addition to posture improvement, may be difficult to don and remove.

Fabrics with the necessary stretch properties often contain synthetic fibers that, in combination with the tight fit, may cause the wearer to overheat. The straps or bands typically attach to a type of panel in the center of the back along the spine, necessitating a counterforce to prevent the shirt from migrating upward due to the pull of the straps. This counterforce may be achieved either by applying a grippy substance or incorporating a belt wrapped around the waist. The integrated straps or bands, and/or the amount of compression, are often not adjustable, potentially making it challenging to achieve a proper and comfortable fit.

Other versions offering proprioceptive orthopedic support may present straps or bands across the chest, as seen in U.S. Pat. No. 9,445,932 to Boynton, and U.S. Pat. No. 9,226,534 to Puni. These designs may be uncomfortable and potentially unflattering for a woman's physique. Another form of shirt or undershirt relies on proprioception or other cues to remind the wearer to pull the shoulders back, without applying tension or force. Examples include U.S. Pat. No. 10,849,779 to Brown et al. and U.S. Pat. No. 10,213,331 to Weller et al.

Achieving healthy posture alignment involves moving the shoulder joints back and down. Existing posture support devices and garments often aim to pull the shoulders back or cue the brain to pull the shoulders back using straps or bands that run along the collarbone midway between the shoulder joint and the neck. This approach may not optimally position the upper body for proper alignment.

Electronic training devices with integrated sensors have been developed to remind users to maintain proper posture. Examples include U.S. Pat. No. 9,406,211 to Sahiholnasab et al., U.S. Pat. No. 10,071,197 to Skelton et al., U.S. Patent Application Publication No. 2019/0183388 to Cohen et al., and U.S. Patent Application Publication No. 2021/0106279 to Cohen et al. These small devices may be adhered to the skin along the spine or elsewhere on the body but provide no physical posture support. They function by monitoring the user's position using sensors and alerting users if they are not in an upright position. These devices may protrude from the skin, potentially making it difficult to wear discreetly. They may also signal the user incorrectly in certain situations, such as when leaning over to empty a dishwasher or pick up an object, potentially limiting the activities that can be performed while wearing the device.

Software-based interventions that interrupt workflow to remind users to correct their posture have also been developed. However, these approaches may interfere with productivity and are often ignored or disabled by users.

In summary, known posture support garment devices may rely on straps over the collarbone, comprise a plurality of elements including semi-rigid and/or inelastic components, require wearing over clothes, resemble compression shirts covering most of the upper body, restrict movement, or fail to maintain their position during use. Furthermore, these devices are typically not connected to a system and method for incentivizing use in exchange for credit that gives users a stake in the system they are participating in, nor do they provide users with full control over their personal data and are not tied to a real-time responsive sensor/haptic system.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

The prior attempts to find solutions that provide for long-term wearability, posture correction, and reduced rigidity has led to suboptimal adherence and limited long-term benefits for users. Therefore, there is an unmet need for a posture support garment that is comfortable for extended wear, allows full range of motion, provides effective posture correction, and can integrate with a system that encourages consistent use through personalized feedback and incentives. The present embodiments address the growing concerns related to poor posture in modern society, including back pain, reduced productivity, and associated healthcare costs.

The present disclosure provides a posture support garment and associated incentive system designed to promote proper posture alignment and encourage consistent use. The garment features a unique structure with a back band, axilla region portions, scapular region portions, underarm regions, and a forward portion that work together to provide biomechanical tension correction and self-stabilizing fit. The garment incorporates sensors, haptic devices, and a monitoring system to collect physiological data, provide personalized feedback, and integrate with a networked incentive system. This system tracks usage, establishes value for participation, and offers exchangeable credits or stakes to users, creating a comprehensive solution for long-term posture improvement and user engagement.

In one embodiment, a posture support garment device is provided. In this embodiment, the posture support garment device includes a back band portion extending across a wearer's back, an axilla region portion connected to the back band portion and extending under the wearer's axilla region, rising above shoulder joints to provide a supportive lift that pulls shoulders back and downward, a scapular region portion joined to the axilla region portion, forming a cross-back support structure, an underarm region joined to the scapular region portion and extending under the axilla on an opposite side from the shoulder, continuing forward along a torso, and a forward portion originating from the underarm regions and fastening in front.

In another embodiment, a system for incentivizing use of a product or device or participation in the system is provided. In this embodiment, the system includes at least one sensor configured to detect usage of a product or device, functional elements configured to collect and analyze usage data, a value establishment mechanism configured to establish a unit of value for usage, an exchange engine configured to exchange said unit of value for credit that has spendable or sellable value in the system or stake, and a flexible system configured to allow expansion of the system to motivate other behaviors, and to include other products, devices and contributions to the system.

In yet another embodiment, a feedback system is provided. In this embodiment, the feedback system includes at least one sensor configured to capture a user signal, at least one haptic device configured to provide sensory feedback to a user, functional elements configured to analyze sensor data locally to extrapolate the user's state, or collect and transmit sensor data to a remote system to extrapolate the user's state and receive instructions, wherein the functional elements are configured to execute instructions locally or from a remote engine to actuate the at least one haptic device to assist the user in tuning voluntary actions with elements of the user's autonomic nervous system to boost, dampen, or maintain a particular physiological state.

In yet another embodiment, a method of incentivizing a user to use a system is provided. In this embodiment, the method includes providing a posture support garment device having onboard sensors for tracking and collecting usage data, establishing a connection with the incentive system through a user interface on an edge device across which data and instructions can be transferred, at predetermined intervals agreed upon by the user through the user interface, collecting sensor data and sharing it with the incentive system, at predetermined intervals, the posture support garment device transmitting data to the incentive system through the user interface about an action recognized by the incentive system to have value, evaluating the usage data, other received data, and applicable terms of use, assessing value and presenting the user an offer in the form of credit or stake understood by the user to have value within the incentive system, settling an exchange agreement if the user accepts the offer either manually or through automation, copying and storing an agreed upon data set and terms into the incentive system, and transferring an agreed amount of the offer to the user's account.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1A illustrates a front view of a posture support garment device, according to aspects of the present disclosure.

FIG. 1B depicts a front view of the posture support garment device of FIG. 1A worn by a wearer, according to an embodiment.

FIG. 2A shows a rear view of the posture support garment device of FIG. 1A, according to aspects of the present disclosure.

FIG. 2B illustrates a rear view of the posture support garment device of FIG. 1A worn by a wearer, according to an embodiment.

FIG. 3A depicts a side view of the posture support garment device of FIG. 1A, according to aspects of the present disclosure.

FIG. 3B shows a side front view of the posture support garment device of FIG. 1A worn by a wearer, according to an embodiment.

FIG. 3C illustrates a side back view of the posture support garment device of FIG. 1A worn by a wearer, according to aspects of the present disclosure.

FIG. 4 depicts a front view of the posture support garment device of FIG. 1A in an alternate configuration, according to an embodiment.

FIG. 5A shows a flat view of the exterior side of the posture support garment device, according to aspects of the present disclosure.

FIG. 5B illustrates another flat view of the exterior side of the posture support garment device, according to an embodiment.

FIG. 5C depicts a flat partial view of the exterior side of the posture support garment device, according to aspects of the present disclosure.

FIG. 5D shows a flat view of the interior side of the posture support garment device, according to an embodiment.

FIG. 5E illustrates adhesive film layers for the posture support garment device, according to aspects of the present disclosure.

FIG. 6A depicts a flat partial view of the interior side of the posture support garment device, according to an embodiment.

FIG. 6B shows a flat partial view of the interior side with an optional hemmed edge, according to aspects of the present disclosure.

FIG. 6C illustrates a flat partial view of the interior side with an optional hemmed sleeve edge, according to an embodiment.

FIG. 7 depicts a flat partial view of the interior side with grippy coating applied, according to aspects of the present disclosure.

FIG. 8A shows a flat partial view of the exterior side with a structural support cover, according to an embodiment.

FIG. 8B illustrates a cross-section of the structural support cover of FIG. 8A, according to aspects of the present disclosure.

FIG. 8C depicts a flat view of the structural support cover with adhesive film, according to an embodiment.

FIG. 9A shows an underarm boning piece, according to aspects of the present disclosure.

FIG. 9B illustrates a top view of an underarm boning piece in a boning encasement, according to an embodiment.

FIG. 9C depicts a top view of two underarm boning pieces in a boning encasement, according to aspects of the present disclosure.

FIG. 9D shows a cross-section of a structural support element, according to an embodiment.

FIG. 10 illustrates a rear view of the posture support garment device with folded sleeve portions, according to aspects of the present disclosure.

FIG. 11 depicts a schematic of components for sensing and actuating functions, according to an embodiment.

FIG. 12 shows a system for incentivizing use of a product such as the posture support garment, according to aspects of the present disclosure.

FIG. 13 illustrates a decentralized governance system for incentivizing product usage, according to an embodiment.

FIG. 14 depicts a method for user interaction with an incentive system, according to aspects of the present disclosure.

FIG. 15 shows a method for collecting and feeding back user signals via sensors and actuators, according to an embodiment.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to a posture support garment device and associated systems for incentivizing use and providing feedback to wearers. The posture support garment device may be designed to improve posture alignment by applying biomechanical tension correction to the wearer's shoulders and back.

The posture support garment device may be constructed from various stretchable materials to provide adjustable support and tension while molding to the wearer's body contours. In some cases, the materials used may include LYCRA®, nylon, rayon, polyester, spandex, and other elastane blends. In other cases, new blended materials or biomaterials such as Sorona® may be utilized in the construction of the posture support garment device.

Manufacturing of the posture support garment device may be accomplished using circular knitting machines to create a seamless continuous piece. This manufacturing method may allow for targeted variation in stretch and support properties across different regions of the garment.

The posture support garment device may be designed with specific stretch characteristics to provide effective posture correction while maintaining comfort. In some cases, the stretch along the direction of the length of the posture garment device may range from 60% to 100% of the material. This stretch property may allow the garment to apply the necessary tension for posture correction while accommodating a range of body movements.

The posture support garment device may incorporate various structural elements and features to provide targeted support and maintain proper positioning on the wearer's body. Additionally, the garment may be equipped with sensors and haptic devices to monitor the wearer's physiological state and provide feedback.

Associated with the posture support garment device, an incentive system may be implemented to encourage consistent use of the garment. This system may track wear time and other usage data, offering rewards or benefits to users based on their participation and engagement with the posture improvement program.

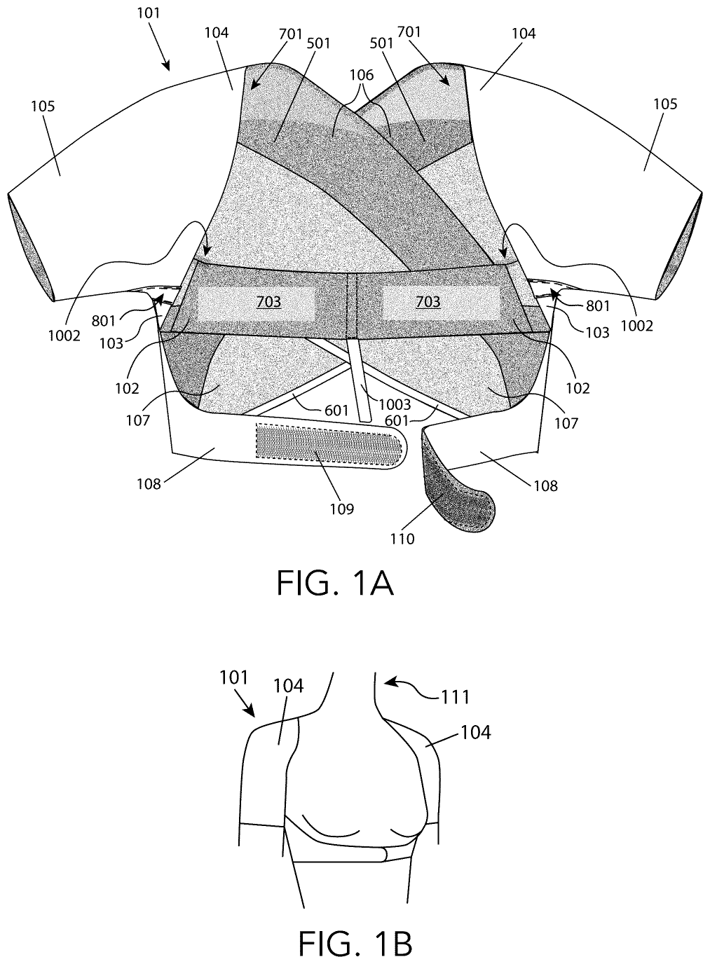

Referring to FIG. 1A, a posture support garment device 101 may be configured to provide biomechanical tension correction to promote proper posture alignment. The posture support garment device 101 includes a back band portion 102 designed to extend horizontally across a wearer's back between the thoracic and sternoxiphoid plane.

An axilla region portion 103 may be connected to the back band portion 102 and extend under the wearer's axilla region. The axilla region portion 103 may rise above shoulder joints 104 and acromial regions to provide a supportive lift that pulls the shoulders back and downward. In some cases, the axilla region portion 103 may include a sleeve member 105 that encircles the shoulder joints 104 and axilla, extending around the deltoid, biceps, and triceps muscles. The sleeve member 105 may be adaptable in length and shape to accommodate various arm sizes and body types.

A scapular region portion 106 may be joined to the axilla region portion 103, forming a cross-back support structure. An underarm region 107 may be joined to the scapular region portion 106 and extend under the axilla on the opposite side from the shoulder, continuing forward along the torso.

A forward portion 108 may originate from the underarm regions 107 and traverse beneath the mammary region from both sides of the wearer's body. The forward portion 108 may include a first fastener 109 on the left side and a second fastener 110 on the right side, allowing the forward portion 108 to fasten securely in front.

The posture support garment device 101 may be constructed from a stretchable material designed to mold to the wearer's body contours and provide adjustable support and tension. In some cases, the stretchable material may include a reinforcement inner layer 501 to enhance support in specific areas.

Referring to FIG. 1B, the posture support garment device 101 may be shown as worn by a wearer 111. The posture support garment device 101 may be designed to provide a self-stabilizing fit that maintains the garment's position during wearer 111 movement without the need for need for external straps. A distinguishing characteristic of the present support garment device 101 is that this pulls back from the shoulder joints, while in many existing posture support devices seek to pull the shoulder back, or cue the brain to pull the shoulders back via proprioception, with straps or bands that run along the collarbone midway between the shoulder joint and the neck, an area of minimal movement with respect to the rest of the body.

In some cases, the posture support garment device 101 may include additional features such as a hemmed edge 601 for improved durability, a reinforcement inner layer grippy coating 701 and a dorsal region grippy coating 703 to enhance garment stability, structural support 903A or 903B to maintain garment sleeve shape with structural support cover cover 801 to keep structural support in place, a sleeve seam 1002 for improved fit, and an alignment tab 1003 to assist in proper positioning of the garment.

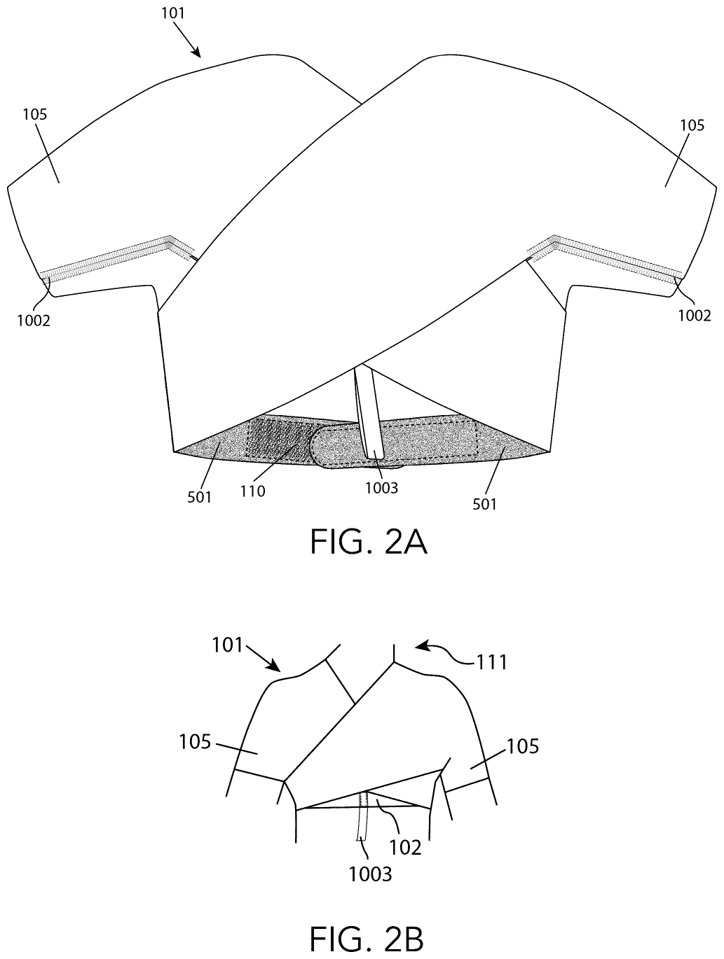

Referring to FIG. 2A, a rear view of the posture support garment device 101 is shown. The posture support garment device 101 may include a back band portion (dorsal area) 102 extending horizontally across the wearer's back. The back band portion (dorsal area) 102 may be designed to provide counter-tension for the straps. The position between the thoracic and sternoxiphoid plane is the most comfortable place to have a trap running horizontally across one's back. (Reference numerals, such as the back band portion 102 are shown in FIG. 2B and additional reference numbers mentioned throughout the specification may be located in different figures relative to the specific one being described in order to be comprehensive in descriptions of an embodiment where more than one view, i.e. shown in a different figure, is helpful to understand the interrelatedness of the elements of the device).

In some cases, the posture support garment device 101 may include a second fastener (right side) 110 positioned at the front of the garment. The second fastener (right side) 110 may be configured to secure the forward portion under the mammary region, as previously described.

The scapular region portion 106 (FIG. 1A) may be joined to the axilla region portion 103 (FIG. 1A), forming an active cross-back support structure. This cross-back support structure may be designed to dynamically distribute tension across the back, potentially assisting in muscle engagement and postural adjustment. The scapular region portion 106 may cross diagonally across the wearer's back, creating a supportive X-shaped pattern.

The sleeve member 105 of the axilla region portion 103 may be visible from the rear view. In some cases, the sleeve seam 1002 may run along the back of the arm, specifically along the long head of triceps brachii. This placement of the sleeve seam 1002 may differ from traditional sleeve seams, which typically run under the arm. The positioning of the sleeve seam 1002 along the back of the arm may potentially contribute to improved comfort and reduced interference with arm movement.

The underarm region 107 may extend from the scapular region portion 106, continuing under the axilla and forward along the torso. This configuration may contribute to the self-stabilizing fit of the posture support garment device 101.

In some cases, the posture support garment device 101 may incorporate additional features not visible from the rear view. These may include the reinforcement inner layer 501, which may provide enhanced support in specific areas. The hemmed edge 601 may be present for improved durability. The reinforcement inner layer grippy coating 701 and dorsal region grippy coating 703 may be incorporated to enhance garment stability against the wearer's skin.

Structural support 903A or 903B may contribute to maintain garment sleeve shape with structural support cover 801 to keep structural support in place of the posture support garment device 101. An alignment tab 1003 may be included to assist in proper positioning of the garment on the wearer's body.

Referring to FIG. 2B, the posture support garment device 101 may be shown as worn by a wearer 111 from the rear view. This view illustrates how the cross-back support structure formed by the scapular region portion 106 and axilla region portion 103 interacts with the wearer's back. The back band portion (dorsal area) 102 may be visible, extending horizontally across the wearer's back between the thoracic and sternoxiphoid plane.

The sleeve member 105 may be seen encircling the wearer's shoulder and upper arm region. The positioning of the sleeve member 105 helps keep the garment device correctly positioned on the body. The length of said sleeve member 105 can vary, from capped sleeves to mid-length to long sleeves extending to or beyond the wrist. A sleeveless configuration is also possible, but is more likely to slip out of place with arm movement.

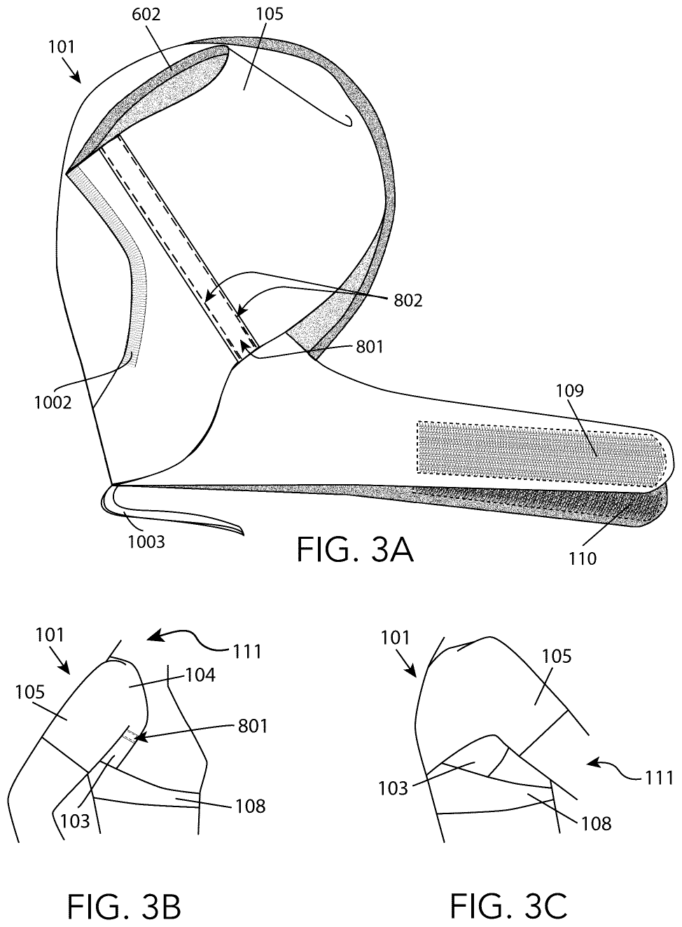

Referring to FIG. 3A, a side view of the posture support garment device 101 may be shown. The posture support garment device 101 may include a shoulder joint 104 area where the axilla region portion 103 extends up and over the wearer's shoulder. A forward portion securing end 108 may be visible, extending from the underarm region 107 towards the front of the garment.

In some cases, the posture support garment device 101 may include a first fastener (left side) 109 and a second fastener (right side) 110. These fasteners may be configured to secure the forward portion securing end 108 in place beneath the wearer's mammary region.

The sleeve member 105 of the axilla region portion 103 may extend around the upper arm area. A sleeve edge 602 may be present at the end of the sleeve member 105, potentially providing a finished edge for improved comfort and durability.

The structural support cover 801 may be visible from the side view, extending along the underarm area. In some cases, reinforcement seams 802 may be incorporated into the structural support cover 801, potentially enhancing the stability and shape retention of this area.

Referring to FIG. 3B, the posture support garment device 101 may be shown as worn by a wearer 111 from a side front view. This view may illustrate how the axilla region portion 103 extends under the wearer's axilla region and rises above the shoulder joint 104, potentially providing a supportive lift that pulls the shoulders back and downward.

The underarm region 107 may be joined to the scapular region portion 106 and extend under the axilla on the opposite side from the shoulder, continuing forward along the wearer's torso.

The forward portion securing end 108 may be visible extending from the underarm region 107 towards the front of the wearer's body. This forward portion securing end 108 may be designed to traverse beneath the mammary region and fasten securely in front.

Referring to FIG. 3C, the posture support garment device 101 may be shown as worn by a wearer 111 from a side back view. This view may illustrate how the axilla region portion 103 and sleeve member 105 interact with the wearer's shoulder and upper arm area.

The back band portion (dorsal area) 102 may be partially visible from this angle, extending horizontally across the wearer's back. The scapular region portion 106 may be seen joining with the axilla region portion 103, potentially forming part of the cross-back support structure.

In some cases, the underarm region 107 may be visible extending from the scapular region portion 106, running under the axilla and continuing forward along the wearer's torso. The forward portion securing end 108 may be seen extending from the underarm region 107 towards the front of the wearer's body.

The side views presented in FIG. 3A, FIG. 3B, and FIG. 3C may illustrate how various components of the posture support garment device 101 work together to provide a supportive and self-stabilizing fit. The combination of the axilla region portion 103, scapular region portion 106, and underarm region 107 may contribute to maintaining the garment's position during a range of wearer 111 movements.

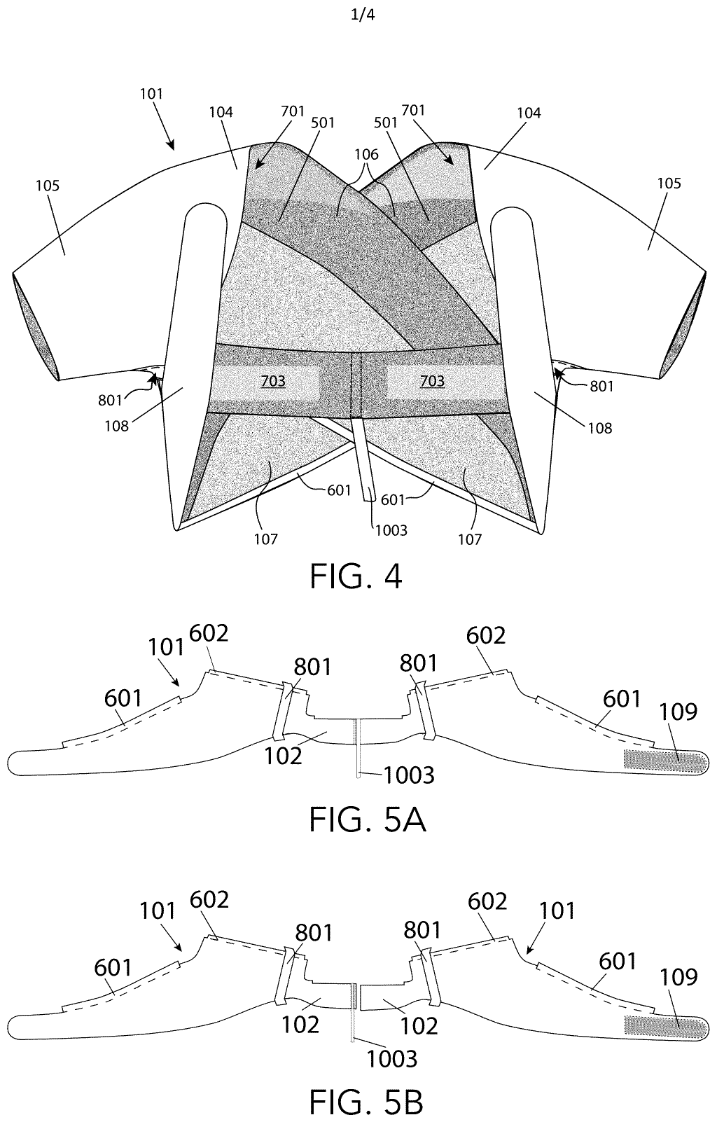

Referring to FIG. 4, an alternate configuration of the posture support garment device 101 may be shown. In this configuration, the forward portion securing end 108 may be positioned differently compared to the primary configuration illustrated in FIG. 1A.

In the alternate configuration, the forward portion securing end 108 may be pulled up towards the deltopectoral triangle region of the wearer's chest. The forward portion securing end 108 may be fastened vertically to the portion of the posture support garment device 101 that covers the pectoralis major muscle on both the left and right sides of the wearer's body.

In some cases, both ends of the posture support garment device 101 may have inside facing, like fasteners. For example, loop fasteners may be used on the inside facing side of the forward portion securing end 108. These loop fasteners may be secured to outside facing fasteners, such as hook fasteners, which may be attached to the outside facing section of the posture support garment device 101 covering the pectoralis major muscle region.

The fasteners used in this alternate configuration may be bonded, sewn, bonded and sewn, or secured by other reliable means to the posture support garment device 101. This fastening method may allow for adjustable tension and support, potentially enabling the wearer 111 to customize the fit and level of postural correction provided by the posture support garment device 101.

In some cases, this alternate configuration may provide different biomechanical tension correction compared to the primary configuration. The upward pull of the forward portion securing end 108 may potentially create a different distribution of forces across the wearer's upper body, which may be beneficial for certain body types or posture correction needs.

The alternate configuration may retain many of the features present in the primary configuration. The back band portion 102, axilla region portion 103, scapular region portion 106, and underarm region 107 may remain in similar positions as in the primary configuration. The sleeve member 105 may continue to encircle the shoulder joints 104 and axilla regions.

In some cases, the alternate configuration may include the reinforcement inner layer 501, which may provide enhanced support in specific areas. The hemmed edge 601 may be present for improved durability. The reinforcement inner layer grippy coating 701 and dorsal region grippy coating 703 may be incorporated to enhance garment stability against the wearer's skin.

The structural support cover 801 may be visible in this alternate configuration, potentially contributing to maintaining the shape of the posture support garment device 101. The alignment tab 1003 may be included to assist in proper positioning of the garment on the wearer's body.

This alternate configuration of the posture support garment device 101 may provide wearers with additional options for achieving a comfortable and effective fit. The ability to adjust the position and tension of the forward portion securing end 108 may allow for personalized posture support based on individual needs and preferences.

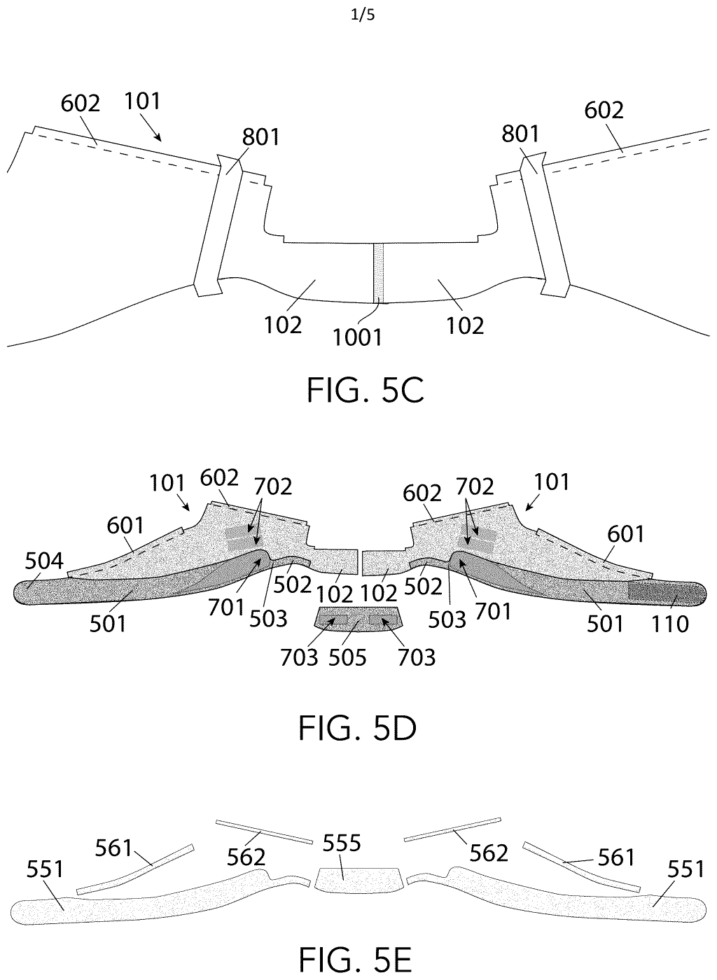

Referring to FIG. 5A and FIG. 5B, flat views of the outside, exterior facing side of the posture support garment device 101 may be shown. In FIG. 5A, the posture support garment device 101 is depicted as a continuous piece of tensile material. In FIG. 5B, the posture support garment device 101 is shown comprising two pieces of tensile material that are mirror reflections of each other.

The back band portion 102 may be designed to extend horizontally across the wearer's back. The first fastener 109 is visible on the right side of the posture support garment device 101. The hemmed edge 601 may be present along the edges of the garment for improved durability. The structural support cover 801 may be visible. The alignment tab 1003 may be included to assist in proper positioning of the garment. The sleeve edge 602 may be present at the end of the sleeve member 105. In FIG. 5A and FIG. 5B, the structural support cover 801 is shown before it is secured garment device and shows the shape of the piece that gets folded over the edges.

Referring to FIG. 5C, a flat partial view of the outside, exterior facing side of the posture support garment device 101 may be shown in isolation. This figure may illustrate an example of two mirrored halves joined by a stitching between mirrored halves 1001. This method of making the posture support garment device 101 by joining mirrored halves may optimize material usage during manufacturing.

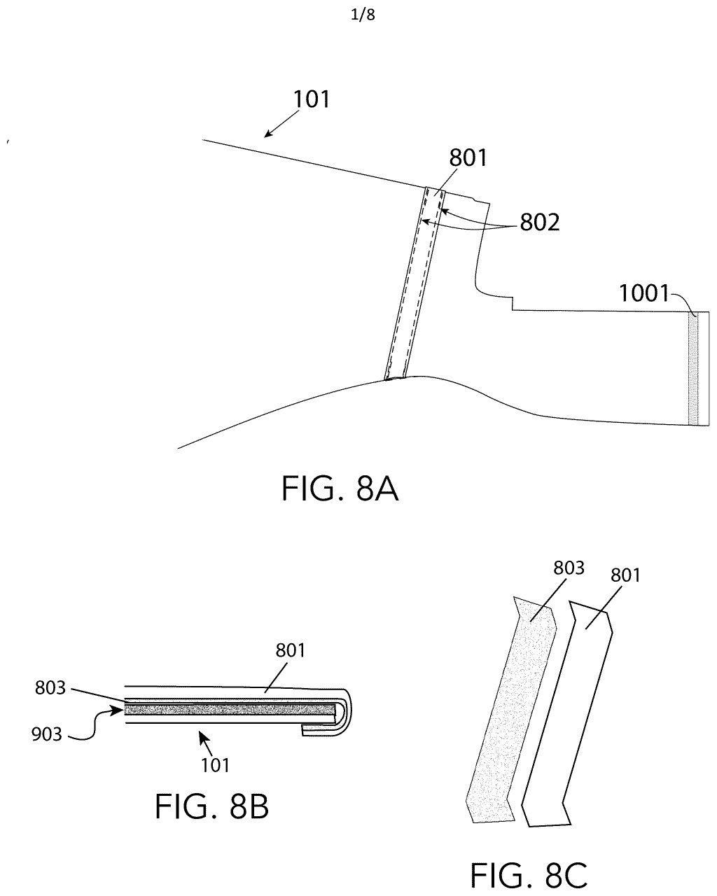

In combination, FIGS. 5A, 5B and 5C show the placement of the underarm structural support cover 801 before it is secured to the garment device 101. The placement, function, composition, and method of securing the structural support cover 801 with seams 802 is detailed in FIGS. 8A, 8B, and 8C.

Referring to FIG. 5D, a flat view of the inside, skin facing side of the posture support garment device 101 may be shown. The posture support garment device 101 may comprise two pieces of tensile material that are mirror reflections of each other, joined at the center of the section that runs across the back band portion 102.

In this view, a partial layer that covers the section of the posture support garment device 101 that runs across the back band portion 102 may be visible. This may be referred to as an inner dorsal reinforcement layer 505. Furthermore, a reinforcement inner layer 501 may be present, following the contour of the posture support garment device 101 that extends along the edge covering the shoulder joint 104.

A narrow region of the reinforcement inner layer 502 may begin where the inner dorsal reinforcement layer 505 ends, running under the axilla, continuing forward, and widening at a shoulder joint section 503. This section may correspond to the shoulder joint 104 region on the outside of the posture support garment device 101. The narrow region 502 may continue diagonally across the scapular region, potentially strengthening the tension force along its trajectory.

In some cases, the posture support garment device 101 may include a shoulder support 504, which may provide additional reinforcement in the shoulder area. The second fastener 110 may be visible on the right side of the garment.

The reinforcement inner layer grippy coating 701 and dorsal region grippy coating 703 may be applied to specific areas of the inner surface to enhance garment stability against the wearer's skin. Additionally, a deltoid region grip coating 702 may be applied to the inside surface of the sleeve member 105 in the area covering the deltoid muscle.

Referring to FIG. 5E, a flat view of various adhesive film layers 551, 555, 561, 562 may be shown. These adhesive layers may be used in the construction of the posture support garment device 101. A support layer adhesive 551 may be used between the outer layer of the posture support garment device 101 and the reinforcement inner layer 501. A dorsal layer adhesive 555 may be used between the outer layer and the inner dorsal reinforcement layer 505.

An edge adhesive 561 may be used to bond the hemmed edge 601, while a sleeve adhesive 562 may be used to bond the sleeve edge 602. These adhesive layers may contribute to a cleaner, more durable construction of the posture support garment device 101.

In some cases, the adhesive substances used may include heat-activated materials such as Polyurethane, Polyamide, Polyester, Polyolefin, and other heat-activated thermoplastics. These adhesives may allow for bonding of layers while maintaining the necessary stretch properties of the posture support garment device 101.

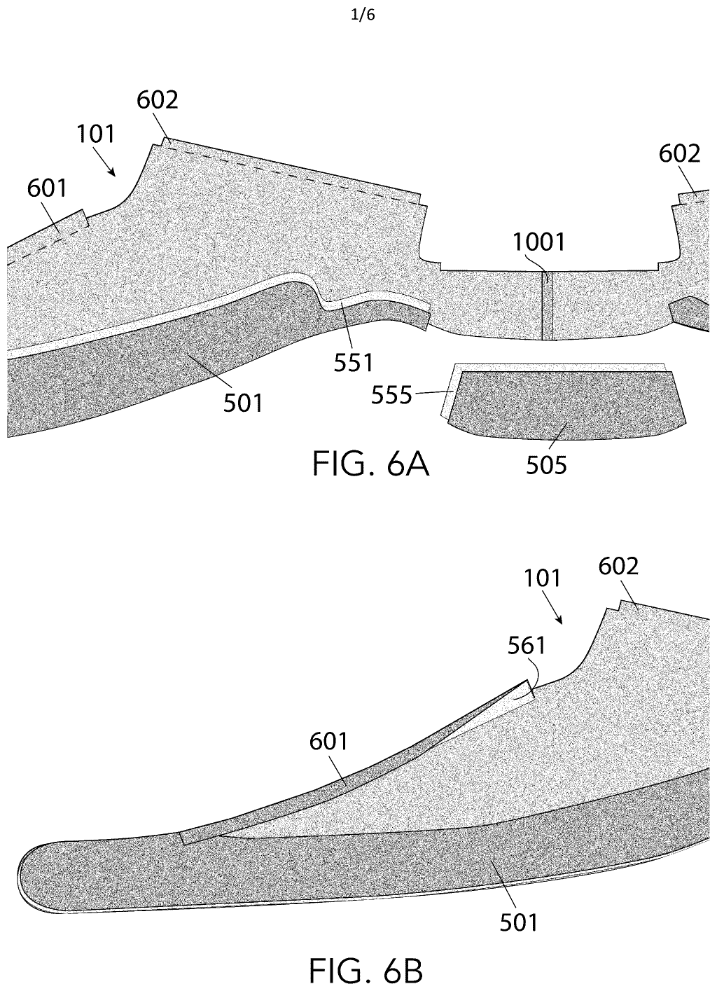

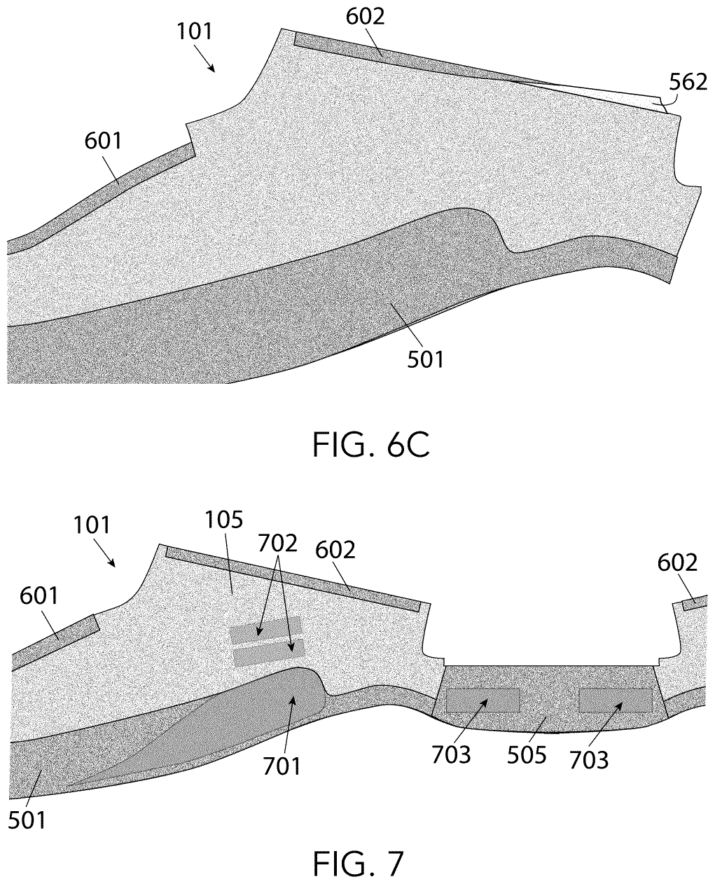

Referring to FIG. 6A, a flat, partial view of the inside, skin facing side of the posture support garment device 101 may be shown in isolation. The posture support garment device 101 may include a reinforcement inner layer 501 and an inner dorsal reinforcement layer 505. These layers may be bonded to the outer layer of the posture support garment device 101 using a support layer adhesive 551 and a dorsal layer adhesive 555, respectively.

In some cases, the posture support garment device 101 may include a hemmed edge 601 and a sleeve edge 602. These edges may contribute to the durability and comfort of the garment. The stitching between mirrored halves 1001 may be visible, potentially indicating a method of construction that optimizes material usage.

Referring to FIG. 6B, a flat, partial view of the inside, skin facing side of the posture support garment device 101 with an optional hemmed edge 601 may be shown. The hemmed edge 601 may run along the edge of the posture support garment device 101 that is opposite to the edge that covers the shoulder joint. In some cases, an edge adhesive 561 may be used to secure the hemmed edge 601, potentially enhancing the durability of the garment.

Referring to FIG. 6C, a flat, partial view of the inside, skin facing side of the posture support garment device 101 with an optional hemmed edge 601 along the sleeve edge 602 may be shown. A sleeve adhesive 562 may be used to secure the sleeve edge 602, potentially contributing to the comfort and durability of the sleeve member 105.

Referring to FIG. 7, a flat, partial view of the inside, skin facing side of the posture support garment device 101 may be shown in isolation, with a grippy coating applied to specific areas. The posture support garment device 101 may include an inner surface with a reversible, repeatable adhesion substance in at least one area to secure the garment against the wearer's body and reduce displacement caused by movement.

In some cases, a reinforcement inner layer grippy coating 701 may be applied to the reinforcement inner layer 501 starting at the shoulder joint 104. A deltoid region grip coating 702 may be applied to the inside, skin facing side of the posture support garment device 101 along the portion of the sleeve member 105 that covers the deltoid muscle. A dorsal region grippy coating 703 may be applied along the inner dorsal reinforcement layer 505 that is horizontally centered across the back band portion 102.

The grippy coatings 701, 702, and 703 may be formulated to provide a reversible, repeatable adhesion to the wearer's skin. These coatings may help keep the posture support garment device 101 in place during various activities and movements. The strategic placement of these grippy coatings may contribute to the overall functionality of the posture support garment device 101 by enhancing its stability and effectiveness in maintaining proper posture alignment.

In some cases, the grippy coatings may be made from substances such as silicone, polypropylene mesh, polyurethane heat-bonding adhesives, heat-activated thermoplastic adhesive films, or other materials with similar adhesive properties. The specific composition of the grippy coatings may be selected based on factors such as durability, comfort, and effectiveness in maintaining garment position.

The combination of the reinforcement layers, adhesive bonding, hemmed edges, and grippy coatings may contribute to the overall performance and comfort of the posture support garment device 101. These features may work together to provide a secure fit, reduce garment displacement, and enhance the posture correction capabilities of the device.

Referring to FIG. 8A, a flat, partial view of the exterior, outside facing side of the posture support garment device 101 may be shown with a structural support cover 801. The structural support cover 801 may be secured to the posture support garment device 101 with reinforcement seams 802. In some cases, the structural support cover 801 may be constructed of the same material as the posture support garment device 101, or any material with similar stretch properties.

Referring to FIG. 8B, there is a cross section which shows the structural support cover 801 on top of the structural support element 903, secured with an optional layer of adhesive film 803, wrapping around the edge of the posture support garment device 101.

Referring to FIG. 8C, a flat view of the structural support cover 801 with the adhesive film 803 may be shown in isolation. The shape and size of the structural support cover 801 may be designed to cover and extend past the structural support element 903 as needed to secure the structural support element 903 and shield the wearer from potential discomfort.

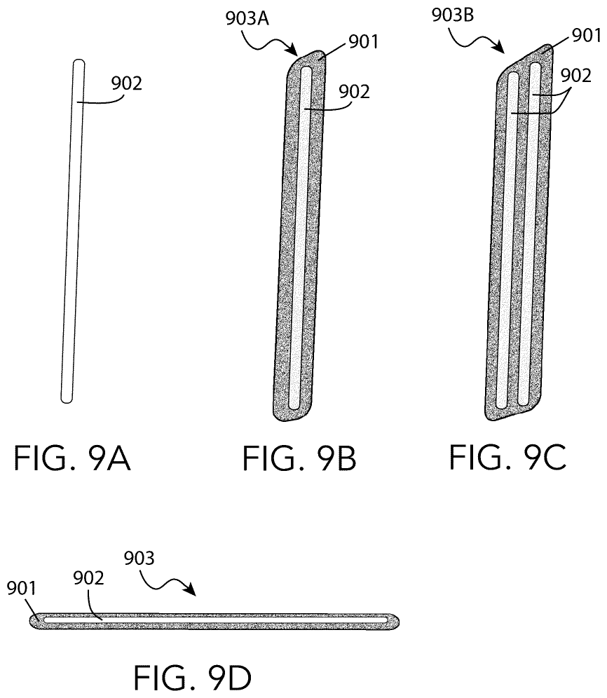

Referring to FIG. 9A, a view of a boning piece 902 may be shown. The boning piece 902 may be a component of the structural support element 903. In some cases, the boning piece 902 may be made of materials with flexible properties, such as metal coil, synthetic whalebone, plastic boning. Other materials may be used in lieu of boning, for example steel string, synthetic materials with properties woven into a mesh.

Referring to FIG. 9B, a top view of the boning piece 902 embedded in a boning encasement 901 may be shown, forming a structural support element 903A. The boning encasement 901 may be a thin, flexible substance that runs at least the length of the boning piece 902 and may be at least twice as wide. The purpose of the boning encasement 901 is to distribute and diffuse the pressure from the boning piece 902.

Referring to FIG. 9C, a top view of two boning pieces 902 running parallel to each other, embedded in the boning encasement 901, may be shown. This configuration may create a structural support element 903B. In some cases, the boning encasement 901 may be made of materials such as silicone, heat activated polypropylene mesh, or heat-activated thermoplastic adhesive films.

Referring to FIG. 9D, a cross section of the structural support element 903 may be shown. In this variation, the boning encasement 901 may be thicker on one side of the boning piece 902 than on the other side. The thicker side of the structural support element 903 may be joined to the exterior, outside facing side of the posture support garment device 101 such that the thicker side faces the wearer's skin. This configuration may reduce pressure from the boning piece 902 under the wearer's arm.

In some cases, at least one flexible structural support element 903 may be integrated within the sleeve member 105 to maintain its shape and fit during movement. The structural support element 903 may comprise at least one flexible boning piece 902 encased in the boning encasement 901, which may be a thin, flexible substance that runs at least the length of the boning piece 902 and may be at least twice as wide.

The structural support element 903 may be designed to prevent the sleeve-like portions of the posture support garment device 101 from bunching under the axilla area, which may cause discomfort and constriction. In some cases, there may be at least one structural support element 903 attached to the outside facing side of the posture support garment device 101 on each sleeve. The structural support can be added topically, as in the present embodiment, or embedded into the posture support garment device 101, for example knit or woven directly into the fabric of the posture support garment device 101. The shape of the structural support may be a variety of shapes and does not need to be a rectangular strip as shown, as it can take any form as long as it fulfills the functional purpose of preventing underarm bunching, comfortably.

The structural support element 903 may extend from the end of the sleeve along the portion of the triceps muscle that runs along the underarm, crossing the latissimus dorsi under the axilla, coming forward and angled slightly down, ending below and next to the pectoralis major muscle, in the vicinity of the top of the serratus anterior muscles. This placement may minimize pressure along the sensitive areas of the underarm and allow for flexibility, while preventing the sleeve member 105 from bunching under the arm.

In some cases, the structural support element 903 may have a thicker side facing the skin and a thinner side facing outward to reduce pressure and bulk. The thinner outside facing side of the boning encasement 901 may reduce the overall thickness of the structural support element 903 so that there may be no unnecessary bulk across the wearer's underarm.

The composition, placement, and function of these structural support elements may contribute to maintaining the shape and effectiveness of the posture support garment device 101. These elements may work together to provide a comfortable and stable fit while allowing for a full range of motion.

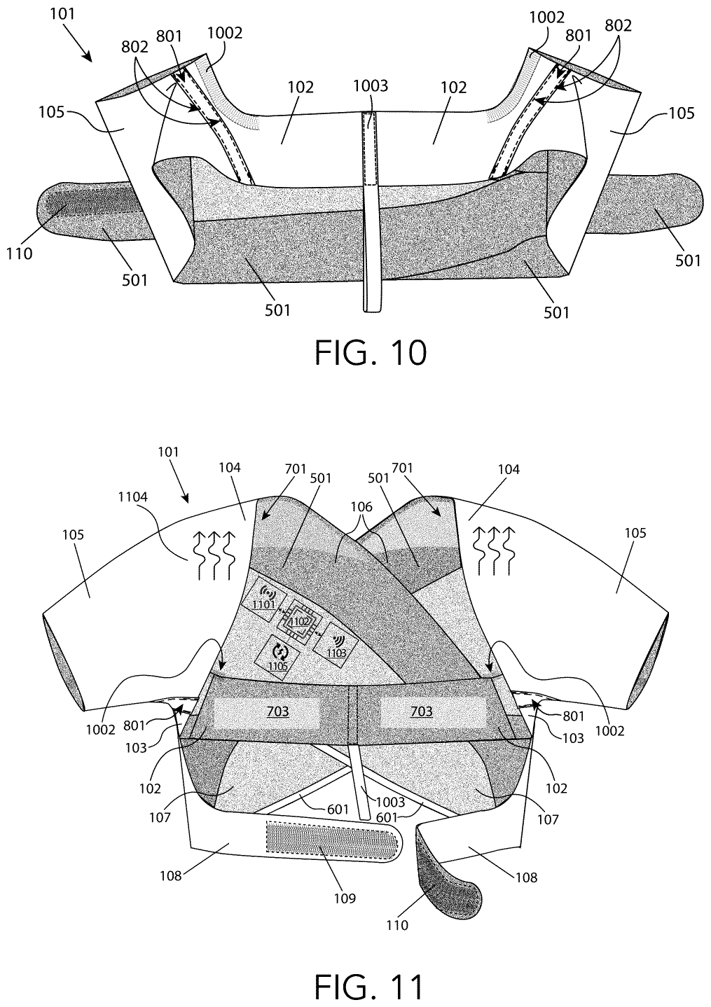

Referring to FIG. 10, a rear view of the posture support garment device 101 may be shown with the sleeve-like portions 105 folded up to reveal the sleeve seams 1002. The sleeve seams 1002 may be joined by sewn stitches, bonding tape, or other methods. Unlike traditional shirt sleeve seams, which typically run along the underarm in the area of the brachii arteries and surrounding nerves, the sleeve seams 1002 in the posture support garment device 101 may be positioned in the dorsal portion of the garment.

The sleeve seams 1002 may start in proximity of the teres major muscle, running along the long head triceps brachii muscle until reaching the end of the sleeve-like portions 105. This placement of the sleeve seams 1002 may potentially contribute to improved comfort and reduced interference with arm movement.

The sleeve-like portions 105 may be form-fitting and may be reinforced by the structural support element 903. The structural support element 903 may be positioned under the structural support cover 801, between the structural support cover 801 and the posture support garment device 101. In some cases, there may be at least one structural support element 903 on each sleeve-like portion 105.

In some cases, the posture support garment device 101 may include additional structural support elements similar to the structural support element 903 throughout the garment, for example surrounding the shoulder area. The purpose of the sleeve-like portions 105 may be to keep the posture support garment device 101 in place, even during vigorous arm movement by the wearer 111.

The back band portion 102 may be visible in FIG. 10, extending horizontally across the wearer's back. The second fastener 110 may also be visible from this rear view. The reinforcement inner layer 501 may provide enhanced support in specific areas of the posture support garment device 101.

In some cases, the posture support garment device 101 may incorporate gussets in regions of the sleeve if the garment is cut and sewn or bonded. These gussets may potentially relieve tightness or constriction when the arm is lifted. If the posture support garment device 101 is knit to form, equivalent relief may be achieved by using knitting yarns with greater stretch properties in regions of tightness, or by knitting additional rows of stitches into the tight regions.

The structural support cover 801 may be visible in this view. Reinforcement seams 802 may be incorporated to enhance the stability and shape retention of the structural support cover 801.

An alignment tab 1003 may be included to potentially assist in proper positioning of the posture support garment device 101 on the wearer's body. The stitching between mirrored halves 1001 may be visible, potentially indicating a method of construction that optimizes material usage.

This sleeve construction and seam placement design of the posture support garment device 101 may contribute to the overall comfort and effectiveness of the device by potentially reducing pressure on sensitive areas, allowing for a full range of motion, and maintaining the garment's position during various activities.

Referring to FIG. 11, the posture support garment device 101 may include a monitoring system for responding to a wearer's physiological state. The monitoring system may comprise various components integrated into the posture support garment device 101.

In some cases, the monitoring system may include at least one sensor 1101 connected to the posture support garment device 101. The sensor 1101 may be positioned at a location appropriate for the sensor's intended function. The sensor 1101 may be designed to continuously or intermittently monitor and gather wearer data. Non-limiting examples of sensor monitoring functions include: measuring temperature, heart rate, position, location, movement, skin contact and pressure, sun exposure, sleep, muscle activity, sweat chemistry, bacteria presence, moisture, garment device material stretch and wear-and-tear, and more. The sensor(s) and corresponding components are part of the garment device system that can sense, store, and conjecture the wearer's state, or discover patterns, by applying artificial intelligence and known machine learning algorithms (for example, is the wearer hot from exercise or a fever), and respond to the wearer based on the synthesized analyzed data set.

The monitoring system may also include functional elements for data storage, onboard processing, communication with other onboard components or offboard edge devices, wireless transmission, and a power source. These functional elements may be operatively linked to the sensor 1101. In some cases, these functional elements may include a microcontroller 1102 with onboard processor and memory.

A wireless interface 1103 may be included in the monitoring system. The wireless interface 1103 may enable communication between the posture support garment device 101 and external devices or systems. Depending on the type of wireless communication, for example if an NFC device is present, a radio antenna may be part of the configuration. When resident energy storage 1105 is necessary, a power supply such as at least one thin-film lithium phosphorous oxynitride battery or lithium-ion battery with plug-in or cable-free (induction or resonance) rechargeable capabilities may be used, or new methods of energy storage that can be packaged in a small, flexible form factor. The components mentioned above may be integrated into, or onto the surface of the garment device 101, or embedded into the thread or yarn itself, or integrated into a flexible, moisture resistant medium, for example but not limited to, a flexible printed circuit board encased in a thin silicone substrate, that can be removed from a pocket-like element on the garment before the garment is washed.

The monitoring system may be configured to collect wearer data through the sensor 1101 positioned to monitor the wearer's physiological state. In some cases, the sensor 1101 and corresponding components may be permanently coupled or infused into the posture support garment device 101. In other cases, the sensor 1101 and components may be removable, for example, via a discrete pocket on the skin-facing side of the posture support garment device 101.

The monitoring system may be configured to wirelessly transmit data to an edge device for further computation. This transmission may occur through the wireless interface 1103. The edge device may be equipped with at least an onboard processor and wireless transmission capability to receive data from the sensor 1101 or the microcontroller 1102 on the posture support garment device 101.

In some cases, the edge device may be configured to perform part of the analytics computation, send data and requests to a remote computational engine and data storage, receive data and instructions from the remote computational engine and data storage, and send data and instructions to the

The monitoring system may be configured to analyze the wearer's state using artificial intelligence and known machine learning algorithms. This analysis may be based on a synthesized data set. In some cases, the analysis may be extended across a wearer population to derive longitudinal insights and offer personalized feedback to the wearer. There are many unique uses of the data analysis that can be incorporated into various embodiments. For example, triangulating what the user is doing based on multiple sensor inputs and responding accordingly-if the user is sweating, with increased heart-rate, is the user having a panic attack, and the haptics should tap the user a breath count to help regulate breathing, or is the user on a run/exercising, and the haptic taps help the runner pump more blood to the brain on foot strike synchronized with heartbeat.

The monitoring system may employ various data synthesis and analysis techniques to derive wearer-specific insights from broader population data. In some cases, the system may utilize data aggregation methods to combine information from multiple wearers, creating a comprehensive dataset that represents a diverse range of posture patterns, body types, and usage scenarios. This aggregated data may be anonymized to protect individual privacy while still providing valuable collective insights.

Machine learning models may be trained on the synthesized dataset to recognize complex relationships between various factors such as wear time, posture changes, physical activity levels, and reported comfort or discomfort. These models may evolve over time as more data is collected, potentially improving their accuracy and predictive capabilities. In some cases, the system may employ transfer learning techniques, allowing insights gained from the broader population to be applied more effectively to new users or those with limited individual data.

The process of deriving wearer-specific insights may involve a combination of population-level analysis and individual data interpretation. The system may first identify general trends and patterns from the broader dataset, then apply these findings to the individual wearer's data to generate personalized recommendations. This approach may allow for more nuanced and contextually relevant insights that take into account both longitudinal population-level trends and individual characteristics.

In some instances, the system may utilize time-based analysis techniques to track changes in posture and related metrics over extended periods. This longitudinal approach may enable the identification of long-term trends and the assessment of the effectiveness of various posture correction strategies over time. By comparing an individual's progress to similar users in the broader population, the system may be able to provide more accurate predictions of expected outcomes and suggest personalized adjustments to improve results.

The synthesis of data may also involve the integration of information from various sensors and data sources beyond the posture support garment device itself. In some cases, the system may incorporate data from other wearable devices, smartphone applications, or external health records to create a more comprehensive picture of the wearer's overall health and lifestyle. This holistic approach may allow for more accurate insights that consider the complex interplay between posture, physical activity, sleep patterns, and other health-related factors.

To ensure the relevance and accuracy of derived insights, the system may employ continuous validation and refinement processes. In some instances, this may involve periodic reassessment of the underlying models and algorithms based on new data and user feedback. The system may also incorporate mechanisms for users to provide input on the accuracy and usefulness of the insights they receive, potentially allowing for ongoing improvement and personalization of the analysis process.

The monitoring system may employ various artificial intelligence and machine learning techniques to analyze the wearer's physiological state and provide personalized insights. In some cases, supervised learning algorithms such as support vector machines (SVMs) or random forests may be used to classify different posture states based on sensor data. These algorithms may be trained on labeled datasets of posture information collected from multiple wearers to improve accuracy across diverse body types and movement patterns.

The monitoring system may employ a variety of advanced artificial intelligence and machine learning techniques to analyze the wearer's physiological state and provide personalized insights. In some cases, deep learning approaches such as convolutional neural networks (CNNs) or recurrent neural networks (RNNs) may be applied to analyze time-series data from the sensors. CNNs may be particularly useful for identifying spatial patterns in posture data, while RNNs may excel at capturing temporal dependencies in movement sequences. These neural network architectures may enable the system to detect subtle changes in posture and movement over time, potentially providing early warnings of developing issues.

In some instances, the monitoring system may utilize federated learning techniques to improve its models while preserving user privacy. This approach may allow the system to learn from data across multiple wearers without directly sharing individual data, potentially addressing privacy concerns while still benefiting from a large, diverse dataset. The federated learning process may involve training local models on individual devices, then aggregating the model updates centrally without transferring raw data.

The system may incorporate anomaly detection algorithms to identify unusual patterns or sudden changes in a wearer's posture or movement. These algorithms may help detect potential injuries, changes in physical condition, or improper use of the garment, allowing for timely interventions or adjustments. For example, isolation forests or one-class SVMs may be employed to detect outliers in the wearer's movement patterns that could indicate a deviation from proper posture.

In some cases, the monitoring system may employ reinforcement learning algorithms to optimize personalized feedback and recommendations for each wearer. These algorithms may learn from the wearer's responses to different interventions and adjust their strategies over time to maximize positive outcomes. For instance, a multi-armed bandit algorithm may be used to determine the most effective times and methods for providing posture correction prompts based on the wearer's individual habits and preferences.

The system may utilize transfer learning techniques to leverage knowledge gained from analyzing data across the wearer population to improve personalized models for individual wearers. This approach may be particularly beneficial for new users, as the system can draw on insights from similar wearers to provide more accurate initial recommendations. For example, a pre-trained neural network on a large dataset of posture information may be fine-tuned on an individual wearer's data to quickly adapt to their specific characteristics.

The monitoring system may implement adaptive learning algorithms that continuously update and refine their models based on new data and feedback. This may allow the system to evolve its understanding of individual wearers and the broader population over time, potentially improving the accuracy and relevance of its insights. For instance, online learning algorithms such as stochastic gradient descent may be used to update model parameters in real-time as new data becomes available.

In some cases, the system may employ multi-task learning techniques to simultaneously analyze multiple aspects of the wearer's physiological state and behavior. This approach may allow the system to leverage correlations between different tasks, potentially improving overall performance and providing more holistic insights. For example, a single neural network may be trained to simultaneously predict, activity level, and comfort, potentially uncovering complex relationships between these factors.

In some cases, hierarchical clustering may be utilized to identify patterns in the wearer's physiological data that may not be immediately apparent. These techniques may help uncover relationships between different aspects of posture, movement, and overall well-being, potentially leading to more comprehensive insights for the wearer.

In some implementations, deep learning approaches such as convolutional neural networks (CNNs) or recurrent neural networks (RNNs) may be applied to analyze time-series data from the sensors. CNNs may be particularly useful for identifying spatial patterns in posture data, while RNNs may excel at capturing temporal dependencies in movement sequences. These neural network architectures may enable the system to detect subtle changes in posture and movement over time, potentially providing early warnings of developing issues.

The monitoring system may incorporate reinforcement learning algorithms to optimize personalized feedback and recommendations for each wearer. These algorithms may learn from the wearer's responses to different interventions and adjust their strategies over time to maximize positive outcomes.

The monitoring system may employ federated learning techniques to improve its models while preserving user privacy. This approach may allow the system to learn from data across multiple wearers without directly sharing individual data, potentially addressing privacy concerns while still benefiting from a large, diverse dataset.

The posture support garment device 101 may include at least one haptic device 1104 embedded in the garment. The haptic device 1104 may be configured to create a responsive, personalized experience for the wearer. The haptic device 1104 may be joined to the posture support garment device 101 at a location corresponding to the haptic device function.

A user interface may be operationally connected to the monitoring system. The user interface may be configured to display information pertinent to the wearer's physiological state and provide controls for the wearer to interact with the monitoring system. In some cases, the user interface may be adapted to accept user input for setting preferences for the operation of the monitoring system and for receiving feedback from the monitoring system based on the wearer's physiological data.

The wireless interface 1103 may be configured to connect to a social element, whereby the wearer can wirelessly receive sensory input from others. This feature may enable social interaction or shared experiences through the posture support garment device 101.

In some cases, the power source for the monitoring system may be onboard the posture support garment device 101. The power source may be removable and/or rechargeable. In other cases, the monitoring system may be powered by a remote host energy source.

The posture support garment device 101 may include yarn engineered to perform sensory, haptic and computational functions. This specialized yarn may be integrated into the fabric of the posture support garment device 101, potentially enhancing its monitoring and feedback capabilities.

The monitoring and feedback system integrated into the posture support garment device 101 may work in conjunction with other components of the garment. For example, the back band portion 102, axilla region portion 103, scapular region portion 106, and underarm region 107 may provide strategic locations for sensor placement or haptic feedback. The sleeve member 105 may incorporate sensors or haptic devices to monitor arm movement or provide feedback related to shoulder positioning.

In some cases, the monitoring system may interact with structural elements of the posture support garment device 101, such as the structural support element 903, to provide targeted feedback or data collection related to posture correction. The reinforcement inner layer 501 or inner dorsal reinforcement layer 505 may potentially house components of the monitoring system, integrating them seamlessly into the garment structure.

The combination of the monitoring and feedback system with the structural design of the posture support garment device 101 may potentially enhance the effectiveness of posture correction and provide valuable data for both the wearer and broader population studies on posture improvement.

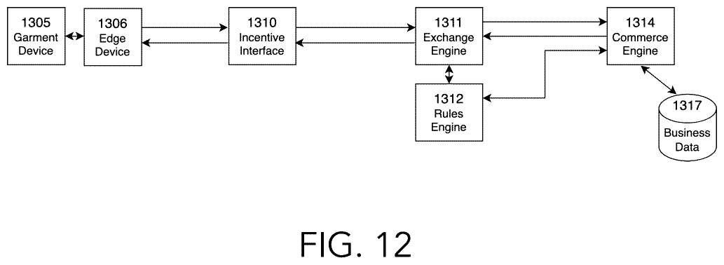

Referring to FIG. 12, an embodiment of a system for incentivizing use of a product. The product may be a posture support garment device 1305 as shown, but this is a non-limiting example embodiment of other products that can be used for the same purpose of the incentive system. The product may include one sensor 1101 configured to detect usage of the product. In some cases, the sensor 1101 may be designed to track wear time, measuring the duration and frequency the product is worn by the wearer 111.

The system may include functional elements configured to collect, transmit, receive, and analyze usage data locally or via a remote computational engine. These functional elements may include an edge device 1306, which may be a mobile phone or other computing device capable of communicating with the garment device 1305.

In some cases, the system may include an incentive system interface 1310 through which the edge device 1306 may communicate with other components of the incentive system. The incentive system interface 1310 may facilitate the transfer of usage data from the garment device 1305 to other parts of the system for analysis and processing.

The system may include an exchange engine 1311 configured to convert usage data into a form of credit or value within the incentive system. A rules engine 1312 may be employed to determine the appropriate conversion rates or rules for translating usage data into credit. To maintain and/or increase the amount of credit/stake, the wearer wears the garment device 1305. If the garment device 1305 is not worn a predefined amount within a preset period of time, an amount of credit/stake may be deducted at a predetermined rate. The value of the credit/stake as a form of currency can fluctuate depending on supply, demand and other factors within the system. A wearer may accrue credit/stake by other forms of participation in the system. While this embodiment illustrates an incentive system to encourage the wearer to wear the posture support garment device 1305, the disclosure is not limited to the garment device. The system for incentivizing use of a product or device in exchange for credit that gives a user a stake in a system the user is participating in can be expanded to motivate other behaviors and generalized to include many products, devices and forms of participation.

In some cases, a commerce engine 1314 may be included in the system to manage transactions and the exchange of credit within the incentive network. The commerce engine 1314 may interact with business data 1317, which may store information about users, their usage patterns, and their accumulated credit.

The system may include a value establishment mechanism configured to establish a unit of value for usage or participation. This mechanism may allow wearers 111 to understand what value to expect in exchange for sharing data and participating in the system. The credit earned through participation may be a form of currency used to store value and may be redeemable within the incentive system.

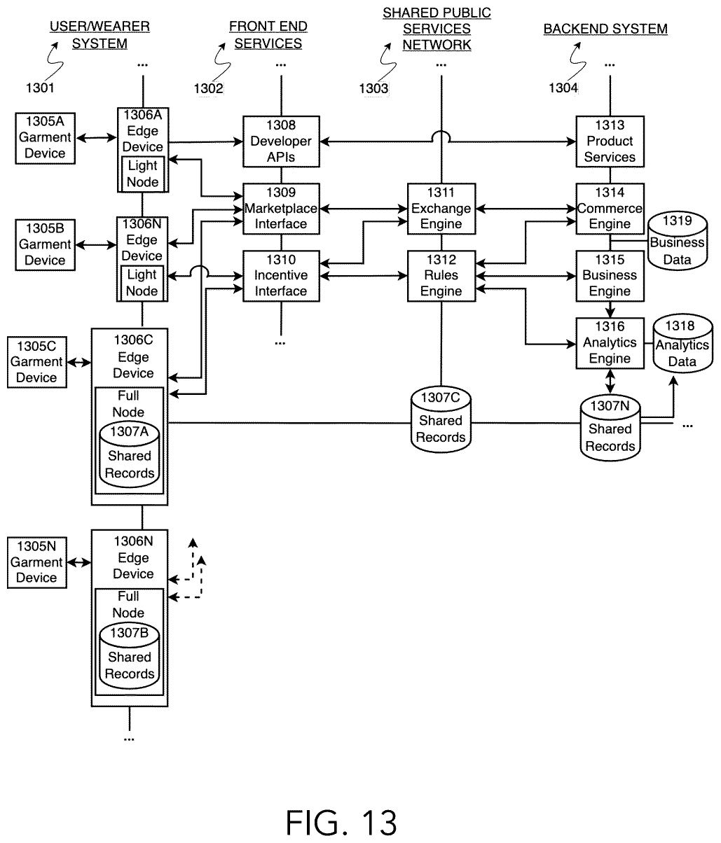

Referring to FIG. 13, an embodiment of a decentralized governance system for incentivizing product or service usage and participation in a product's economic system may be shown. This system may be integrated with the posture support garment device 101 to track wear time, reward participation, and facilitate user engagement based on detected duration and frequency of use. Non-limiting example applications using sensor data include: wear-time data may be extrapolated from skin temperature, or presence of heartbeat, combined with other sensor readings. Wear-time attribution may be time based, or based on other quantifiable increments, for example number of heartbeats. In addition to awarding or deducting credit/stake depending on wear-time as described in FIG. 12, wear-time combined with other data can be used by an analytics engine 1316 and analytics database 1318 to ascertain the condition and durability of the posture garment device 1305C over time, which could inform future posture support garment device 1305A . . . N development. Skin temperature data can be passed to the analytics engine 1316 and analytics database 1318 to analyze individual and aggregate temperature over time, and synthesized with a broader data set to conjecture the wearer's state or discover patterns, by applying artificial intelligence and machine learning algorithms. The result of the analysis can generate a response instruction or recommendation, or an insight that is passed back to the edge device 1306C, for example if the wearer is cold, an instruction could be to warm the wearer to a preferred skin temperature by activating heat elements in the yarn of the posture garment device 1305C. While these examples reference skin temperature, there are many possible composite metrics and observations that can be derived from a combination of measures from sensors and other inputs.

The system may include a user system 1301, which may comprise multiple garment devices 1305A, 1305B, 1305C, and 1305N, each associated with a different wearer 111. These garment devices may communicate with corresponding edge devices, which may be configured as light nodes 1306A, full nodes 1306C, or other types of nodes 1306N within the network.

In some cases, the system may employ a shared public services network 1303, which may include components such as the exchange engine 1311, rules engine 1312, and other services accessible to all participants in the network. Front end services 1302 may provide interfaces for users to interact with the system.

The system may utilize shared records 1307A, 1307B, 1307C, and 1307N, which may be implemented using blockchain technology for secure, decentralized data storage and transactions. This decentralized structure may allow multiple entities to participate without a singular controlling authority.

In some cases, the system may include a backend system 1304 comprising various components such as a business engine 1315, analytics engine 1316, and databases for analytics data 1318 and business data 1319. These components may work together to process usage data, manage transactions, and derive insights from user participation.

The system may include an exchange system configured to exchange the unit of value for spendable or tradable credit within the incentive network. In some cases, this credit may be a stake, a form of share or asset that can hold value and be traded between parties within the user system 1301.

A marketplace interface 1309 may be provided, allowing users to exchange their earned credit for goods, services, voting on company decisions like new features, or additional functionality related to the posture support garment device 101 or any other product. In addition. Developer APIs 1308 may be available, enabling third-party developers to create new applications or services that integrate with the incentive system. Credit retained by the wearer may be issued as, or converted to, a form of currency which the wearer can spend, for example but not limited to: to vote on product or company decisions, to unlock more functionality in the posture support garment device, to purchase a new garment support device, convert to shares of the parent company of the garment device, and more. The system for incentivizing use of a product or device in exchange for credit that gives a user a stake in a system the user is participating in can be expanded to motivate other behaviors and generalized to include many products, devices and forms of participation.

The flexible framework may incorporate a standardized integration protocol that allows for seamless connection of various external devices and data sources. This protocol may utilize APIs and standardized data formats to facilitate easy integration of new devices, sensors, or applications. For example, a participant in the user system 1301 may contribute code that enables new functionality in a garment device by creating software using developer APIs 1308 and publishing the code through the marketing interface 1309 coupled to a non-interchangeable unit (a unique token) establishing ownership of the asset such that other participants in the user system 1301 can purchase rights to use.

The flexible framework may also include a user-configurable rules engine that allows for the creation of custom incentives and goals based on data from multiple devices or sources. Users may be able to set up personalized challenges that combine posture metrics with other health-related data, such as steps taken, hours of sleep, or even dietary information. This multi-faceted approach may provide a more holistic view of the user's health and encourage broader lifestyle improvements beyond just posture correction.

In some cases, the system may incorporate a federated learning approach that allows for the integration of insights from multiple devices while preserving user privacy. This approach may enable the system to learn from aggregated data across various devices and users without directly sharing individual data, potentially improving the accuracy and relevance of posture recommendations while maintaining data security.

The framework may include a dynamic feedback mechanism that can adjust the type and frequency of feedback based on data from multiple sources. For example, if data from a connected sleep tracker indicates poor sleep quality, the system may adjust its posture correction prompts to be less frequent or intense during the following day, potentially adapting to the user's overall well-being and energy levels.

In some implementations, the system may provide a virtual environment or digital twin capability that integrates data from multiple devices to create a comprehensive representation of the user's posture and movement patterns. This virtual model may allow for more sophisticated analysis and visualization of posture issues, potentially enabling more targeted and effective interventions.

The flexible framework may also include a social integration component that allows users to connect with others using similar devices or working towards similar posture goals. This feature may enable the creation of challenges, competitions, or support groups that span across different devices and data sources, potentially increasing user engagement and motivation.

In some cases, the system may incorporate an AI-driven recommendation engine that can suggest new devices or data sources that may be beneficial for a user's specific posture improvement goals. This engine may analyze the user's current data and progress, compare it with anonymized data from other users, and suggest additional devices or contributions that could enhance the effectiveness of their posture improvement efforts.

In some cases, the system is configured to extend incentivization to additional behaviors and integrate other devices or contributions. This flexibility may allow the system to evolve over time, potentially incorporating new types of wearable devices or health-related activities into the incentive structure.

The flexible framework for extending incentivization may be implemented as a modular, extensible system that allows for the integration of various data sources, devices, and behaviors beyond the initial posture support garment device. This framework may utilize a plugin architecture, enabling seamless addition of new components without requiring significant modifications to the core system.

In some cases, the flexible framework may include an API layer that allows third-party developers to create custom integrations. This API may provide standardized methods for data input, processing, and output, ensuring compatibility across different devices and data types. The framework may also incorporate a data normalization layer to convert diverse inputs into a standardized format for consistent processing and analysis.

The system may include a configurable rules engine that allows administrators or users to define custom incentivization rules based on various parameters. For example, users may be able to create personalized goals or challenges, with the system automatically tracking progress and awarding incentives upon completion.

The system may also incorporate a feedback loop mechanism, continuously evaluating the effectiveness of different incentivization strategies and automatically adjusting reward structures to optimize user engagement and behavior change outcomes. This adaptive approach may help maintain long-term user interest and participation in the incentivization program.

In some cases, the decentralized nature of the system may provide users with greater control over their data and participation. The use of blockchain or blockchain-like technology for shared records may enhance security and transparency within the system, potentially increasing user trust and engagement.

The combination of tracking and earning credit for wear-time, converting credit into spendable value in the described system, and a decentralized governance structure may create a comprehensive system for encouraging consistent wear and use of products, or garments such as the posture support garment device 101. In the case of the posture garment device, this system may potentially promote long-term posture improvement by rewarding regular wear and active participation in the broader health ecosystem.

The decentralized nature of the system may allow for distributed governance and participation without relying on a single centralized authority. This approach may leverage blockchain technology and smart contracts to create a trustless environment where multiple entities can interact, contribute, and benefit from the incentive system.

In some implementations, the system may utilize a consensus mechanism such as proof-of-stake or delegated proof-of-stake to validate transactions and maintain the integrity of the shared records. This may allow participants to have a say in the governance of the system proportional to their stake or level of participation. Users who consistently wear the posture support garment device 101 and actively engage with the system may earn voting rights or governance tokens, potentially giving them more influence in decision-making processes.

The system may incorporate decentralized autonomous organizations (DAOs) to manage certain aspects of the incentive program. These DAOs may be governed by smart contracts that automatically execute based on predefined rules and user inputs. For example, a DAO may be responsible for managing the distribution of rewards, adjusting incentive parameters, or approving new features for the posture support garment device 101. This structure may allow for community-driven development and decision-making without the need for a central authority.

In some cases, the system may employ a federated architecture, where multiple independent nodes operate together to form the network. Each node may represent a different entity, such as a healthcare provider, fitness center, or community organization. These nodes may maintain their own local data and rules while still participating in the broader network. This approach may allow for greater flexibility and customization at the local level while still benefiting from the network effects of the larger system.

The system may utilize decentralized identity (DID) solutions to manage user authentication and data privacy. Users may have control over their own digital identities and choose which information to share with different parts of the network. This decentralized approach to identity management may enhance privacy and security while still allowing for seamless interactions within the incentive system.

In some implementations, the system may incorporate decentralized finance (DeFi) principles to create a more open and accessible economic model. This may include features such as decentralized exchanges for trading incentive tokens, liquidity pools to ensure token availability, and yield farming mechanisms to reward long-term participation. These DeFi elements may provide additional incentives for users to engage with the system and may create new opportunities for value creation within the ecosystem.

The system may leverage decentralized storage solutions such as InterPlanetary File System (IPFS) to store and distribute data across the network. This approach may enhance data resilience and availability while reducing reliance on centralized servers. Users may have the option to store their personal data on their own nodes, giving them greater control over their information while still allowing for aggregated, anonymized data analysis to improve the overall system.

In some cases, the system may incorporate zero-knowledge proofs or other privacy-preserving technologies to allow for verification of user actions or achievements without revealing sensitive data. This may enable users to prove their adherence to posture improvement goals or participation in challenges without compromising their privacy. Such mechanisms may foster trust in the system while maintaining the confidentiality of individual user data.

The decentralized governance structure may also allow for the creation of sub-networks or specialized communities within the larger ecosystem. These sub-networks may focus on specific aspects of posture improvement, health conditions, or demographic groups. Each sub-network may have its own set of rules and incentives while still interoperating with the broader system. This modular approach may enable greater customization and targeted interventions while maintaining the benefits of the larger decentralized network.

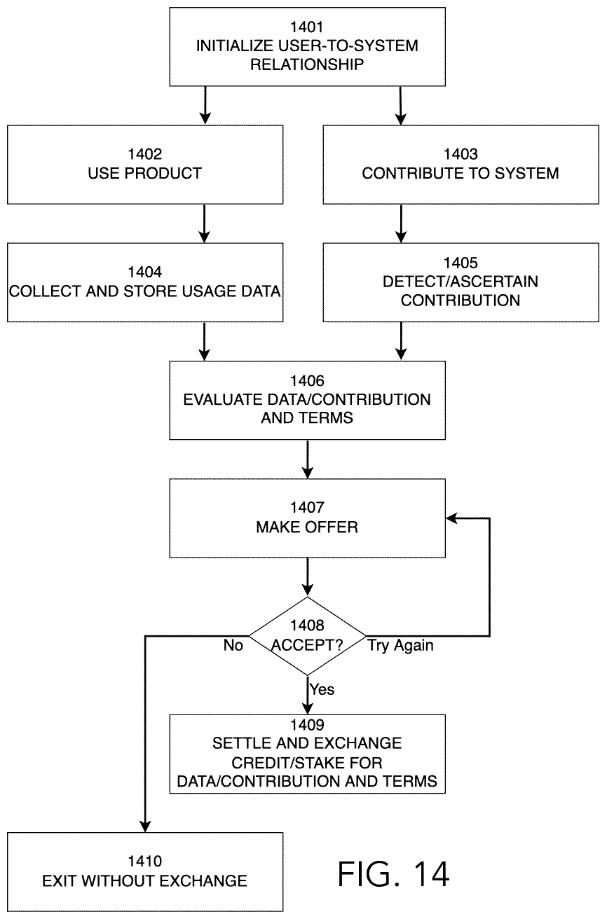

Referring to FIG. 14, a method for a wearer 111 to interact with an incentive system to exchange user data or contributions for credit or stake may be illustrated.

The method may begin with an initializing user-to-system relationship step 1401. In this step, a wearer 111 may put on a posture support garment device 101, establish a wireless connection between the posture support garment device 101 and an edge device 1306, and access an interface on the edge device 1306. Through this interface, the wearer 111 may be onboarded, presented with agreements, and have a user identity and account established. Usage and contribution tracking may also be enabled during this step.

Following initialization, a use product step 1402 may occur. During this step, the wearer 111 may wear the posture support garment device 101. The posture support garment device 101 may include onboard sensors 1101 for tracking and collecting usage data of the posture support garment device 101.

Concurrent with the use product step 1402, a collect and store usage data step 1404 may take place. The sensors 1101 on the posture support garment device 101 may collect data, which may be sent wirelessly to a repository in the wearer's control. This data collection may occur in real-time or may be stored onboard the posture support garment device 101 and sent intermittently to the wearer's repository.

In some cases, a contribute to system step 1403 may occur. The wearer 111 may contribute to the system by sharing product experiences or making referrals through an interface. A detect/ascertain contribution step 1405 may enable the system to detect these contributions.

An evaluate data/contribution and terms step 1406 may follow. The system may evaluate the wearer's data or contribution and the wearer's terms of use. This evaluation may occur in real-time, when certain thresholds are met, or at other frequencies.

Based on this evaluation, a make offer step 1407 may occur. The system may assess the value of the data or contribution and present an offer to the wearer 111. This offer may be in the form of credit or stake understood by the wearer 111 to have value within the incentive system.

An acceptance step 1408 may follow if the wearer 111 accepts the offer. If the wearer 111 accepts, a settle and exchange credit/stake for data/contribution and terms step 1409 may occur. In this step, the agreement may be settled, and the exchange may be made. The wearer's data or contribution may be ingested and stored by the system, and the wearer's account may be credited or the stake amount may be transferred to the wearer's account.

In some cases, the credit or stake may be in the form of digital tokens that can be exchanged for goods, services, voting on product features, or unlocking additional functionality within the incentive system. These digital tokens may represent ownership of digital assets or encode specific usage rights.

If the wearer 111 does not accept the offer, the wearer 111 may either make a counteroffer or ask the system to make another offer. If no agreement is reached, an exit without exchange step 1410 may occur, where either the wearer 111 or the system may exit the negotiation without an exchange being made.

The method may involve collecting sensor data at certain intervals agreed upon by the wearer 111 through the user interface, and sharing this data with the incentive system. The posture support garment device 101 may also transmit data about an action recognized to have value to the incentive system through the user interface.

This method may provide a framework for incentivizing use of the posture support garment device 101 and participation in the broader incentive system, potentially encouraging consistent wear and engagement with posture improvement efforts.

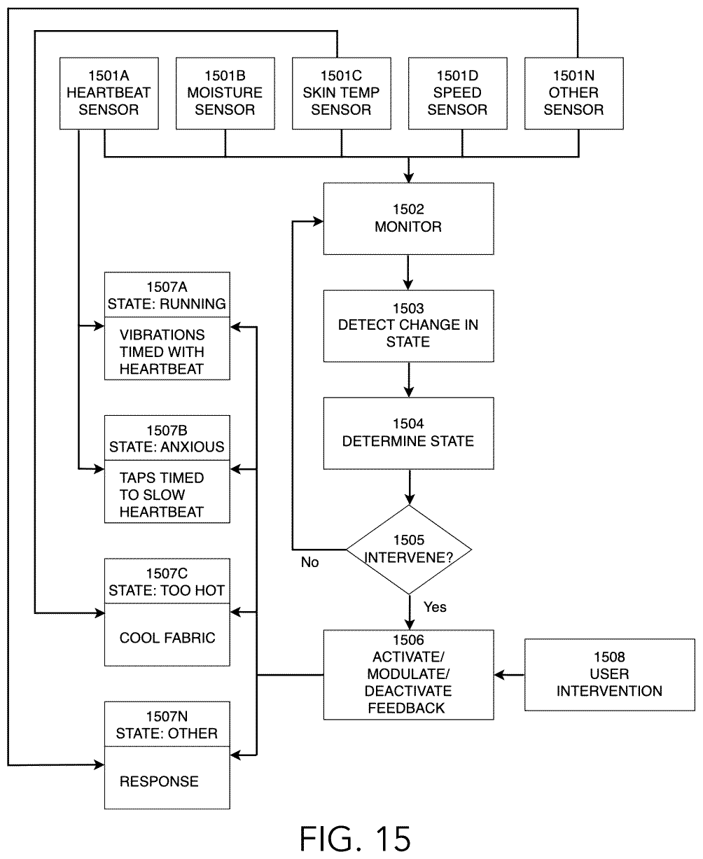

Referring to FIG. 15, a feedback loop mechanism for collecting a wearer's signal via sensors and providing feedback through haptics or actuators to regulate the wearer's physiological state may be illustrated.

The feedback loop mechanism may include various types of sensors for monitoring different physiological parameters of the wearer. In some cases, a heartbeat sensor 1501A may be used to detect the wearer's heart rate. A moisture sensor 1501B may be employed to measure the wearer's perspiration levels. A skin temperature sensor 1501C may be utilized to monitor the wearer's body temperature. A speed sensor 1501D may be used to track the wearer's movement or activity level. In some cases, other sensors 1501N may be incorporated to measure additional physiological or environmental parameters.

The feedback loop mechanism may begin with a monitor step 1502, where the sensors continuously or intermittently collect data from the wearer. This data may be processed and analyzed in real-time or at predetermined intervals.

Following the monitor step 1502, a detection step 1503 may occur. During this step, the system may identify any significant changes or patterns in the collected sensor data. For example, the detection step 1503 may recognize an increase in heart rate, a rise in skin temperature, or a change in movement speed.

After the detection step 1503, a determine state step 1504 may take place. In this step, the system may analyze the detected changes across multiple sensors to infer the wearer's current physiological state. The system may employ artificial intelligence and machine learning algorithms to interpret the data and determine the most likely underlying cause of the new state.

Based on the determined state, a decision step 1505 may follow. During this step, the system may evaluate whether a response should be activated, modified, or deactivated. This decision may be based on predefined parameters set by the wearer and the available sensory feedback mechanisms of the

If the decision step 1505 determines that a response is necessary, an active/modulate/deactivate feedback step 1506 may occur. This step may involve activating, adjusting, or turning off various haptic or actuator devices embedded in the posture support garment device 101.

The feedback provided may be tailored to different physiological states. For example, triangulating from multiple sensors' data, it may be determined the wearer is running for exercise 1507A, the system may synchronize haptic vibrations with the wearer's heartbeat to potentially assist in coordinating foot strike with heart rate to increase blood flow to the brain. If instead the data collected and analyzed across the sensors indicates a state of anxiety 1507B, the haptic devices may generate pulsations or taps to help the wearer regulate breathing and potentially reduce anxiety. A temperature state 1507C may trigger cooling or warming mechanisms in the posture support garment device 101 to help maintain optimal body temperature. Other states 1507N may elicit different types of feedback responses as appropriate. A feedback loop mechanism may act as an amplifier or dampener to regulate a state of well-being by helping the user tune voluntary actions with the user's autonomic nervous system, which controls involuntary actions, can be expanded to other actions, and generalized to include many products and devices. In another example, the system may track the wearer's heart-rate via sensor 1101 and emit a signal to the haptic transducers 1104 to generate a cue, a small vibrating pulse in time with the wearer's heartbeat. The wearer can use this cue to synchronize foot strike with heartbeat, which has been shown to increase blood flow to the brain, increasing energy and improving brain performance.

In some cases, manual user intervention 1508 may be possible at any point in the feedback loop. The wearer may have the ability to manually activate, modulate, or deactivate feedback mechanisms through a user interface connected to the monitoring system.

The feedback loop mechanism may work in conjunction with other components of the posture support garment device 101. For example, the sensors and haptic devices may be strategically placed in areas such as the back band portion 102, axilla region portion 103, or scapular region portion 106 to optimize data collection and feedback delivery.

In some cases, the feedback loop mechanism may interact with the structural elements of the posture support garment device 101. For instance, the structural support element 903 may house certain sensors or haptic devices, integrating them seamlessly into the garment structure.

The data collected and processed by the feedback loop mechanism may be subject to a data management system. This system may allow the wearer to control access, usage, privacy, and security of their personal data collected by the monitoring system. The wearer may have the ability to set preferences for data sharing, storage, and deletion through a user interface connected to the monitoring system.

In some cases, the data management system may interact with the shared records 1307A, 1307B, 1307C, and 1307N of the decentralized governance system described earlier. This interaction may ensure that the wearer's data privacy preferences are respected across the broader incentive system while still allowing for anonymized data analysis to improve the overall effectiveness of the posture support garment device 101.

The feedback loop mechanism, in combination with the data management system, may provide a comprehensive approach to monitoring and improving the wearer's physiological state while respecting their privacy and data control preferences. This system may potentially enhance the effectiveness of the posture support garment device 101 in promoting proper posture and overall well-being.

### Definitions

The term “posture support garment device” as used herein refers to a wearable garment designed to improve and maintain proper posture alignment through biomechanical tension correction and supportive structures. This device may include various components such as a back band portion, axilla region portion, scapular region portion, underarm region, and forward portion, working in concert to provide postural support.

The term “back band portion” as used herein refers to a horizontal section of the posture support garment device that extends across a wearer's back, specifically positioned between the thoracic and sternoxiphoid plane. This portion serves as a foundational element for the overall structure and support provided by the garment.

The term “axilla region portion” as used herein describes a section of the posture support garment device that connects to the back band portion and extends under the wearer's axilla (armpit) region. This portion is designed to rise above the shoulder joints and acromial regions, providing a supportive lift that pulls the shoulders back and downward to promote proper posture alignment.

The term “scapular region portion” as used herein refers to a component of the posture support garment device that is joined to the axilla region portion. This portion forms an active cross-back support structure that dynamically distributes tension across the back, assisting in muscle engagement and postural adjustment.

The term “underarm region” as used herein describes a section of the posture support garment device that is joined to the scapular region portion and extends under the axilla on the opposite side from the shoulder. This portion continues forward along the torso, contributing to the self-stabilizing fit of the garment during wearer movement.

The term “forward portion” as used herein refers to a section of the posture support garment device that originates from the underarm regions and traverses beneath the mammary region from both sides of the wearer's body. This portion is designed to fasten securely in front, completing the supportive structure of the garment.

The term “sleeve member” as used herein describes a component of the axilla region portion that encircles the shoulder joints and axilla, extending around the deltoid, biceps, and triceps muscles. This member is adaptable in length and shape to accommodate various arm sizes and body types.

The term “structural support element” as used herein refers to a flexible component integrated within the sleeve member of the posture support garment device. This element is designed to maintain the shape and fit of the sleeve during movement, mitigating material bunching in the underarm region.

The term “reversible, repeatable adhesion substance” as used herein describes a material applied to the inner surface of the posture support garment device in specific areas. This substance is designed to secure the garment against the wearer's body and reduce displacement caused by movement, while allowing for repeated application and removal.

The term “monitoring system” as used herein refers to a set of components integrated into the posture support garment device that collect, process, and analyze data related to the wearer's physiological state and garment usage. This system may include sensors, onboard functional elements for data processing and storage, and wireless transmission capabilities.

The term “haptic device” as used herein describes a component embedded in the posture support garment device that is capable of providing tactile feedback to the wearer. This device is configured to create a responsive, personalized experience based on data collected by the monitoring system.

The term “edge device” as used herein refers to a computing device, such as a smartphone or tablet, that serves as an intermediary between the posture support garment device and remote computational resources. This device may perform initial data processing and facilitate communication with broader network systems.

The term “incentive system” as used herein describes a network-based framework designed to encourage consistent wear use of products and garments such as the posture support garment device and participation in the system. This system may include mechanisms for tracking wear-time, assigning value to participation, and exchanging earned credits for various benefits.

The term “wear-time” as used herein indicates the dates-and-times, frequency, duration, cumulative amount of time, or other quantifiable increments capable of being attributed to a product or device such as the posture support garment device being worn by a wearer.

The term “credit” or “stake” as used herein refers to a form of value earned within the incentive system through wear of the posture support garment device or other recognized contributions. This value may be redeemable for goods, services, voting rights, or additional functionality within the system, or may represent a form of ownership or voting rights in system governance.

The term “decentralized governance system” as used herein describes an organizational structure for the incentive system that allows multiple entities to participate without a singular controlling authority. This system may utilize technologies such as blockchain to ensure transparency, security, and user control over personal data.

### Advantages

The posture support garment device described herein may offer several advantages over prior art solutions. Unlike traditional posture correction devices that rely on rigid components or external straps, this garment may provide biomechanical tension correction through a flexible, self-stabilizing design. The integration of axilla region portions, scapular region portions, and underarm regions may work in concert to create a dynamic support structure that adapts to the wearer's movements while maintaining proper posture alignment. This approach may allow for a more comfortable and effective posture correction experience compared to static or restrictive designs.

In contrast to Brown's device, which primarily focuses on applying pressure through diagonal tension straps, the present posture support garment device may utilize a more comprehensive approach to posture correction. The garment's design may incorporate multiple interconnected regions that work together to provide targeted support and tension distribution across the wearer's upper body. This may result in a more nuanced and adaptable posture correction system that can accommodate a wider range of body types and movement patterns. Additionally, the self-stabilizing fit of the present device may eliminate the need for frequent adjustments or external fastening mechanisms, potentially improving user compliance and long-term effectiveness.

In addition, Brown discloses a chest portion and a stomach and waist portion, and is worn similarly to wearing compression shirt with straps attached to it. In Brown, the tension forces are fundamentally different. In the present embodiment, the posture garment does not have chest and stomach or waist portions, and the device creates a closed loop that does not rely on a very tight fitting shirt, which would not only be hot, but very uncomfortable for women. The embodiments disclosed in Brown either must to be worn very tight across the upper body, or will displace with any degree of movement.

Lastly, with respect to the disclosures in Brown, the pieces of the device are bulky, and thus are not intended to be worn under any piece of clothing. In the present embodiments, the posture garment is seamless and meant to be worn against the skin under clothing, and present no lumps and bumps under a fitted shirt, for example.

While Zoll's wearable medical device focuses on cardiac monitoring for cardiac arrest using ECG electrodes for medical purposes, the posture support garment device described herein may extend beyond simple physiological monitoring to provide real-time feedback to the user depending on analyzing sensor and other data to determine state and/or activity. The integration of AI-driven analysis across sensor inputs and personalized haptic feedback may represent a significant advancement over traditional monitoring systems. By continuously analyzing the wearer's condition and providing real-time guidance through tactile cues, the device may offer a more interactive and personalized approach to overall health and well-being. This feature may help wearers develop better habits over time, potentially leading to more sustainable long-term results.

Furthermore, the posture support garment device may incorporate a sophisticated data management system that prioritizes user privacy and control. Unlike many existing wearable devices that may collect and transmit data without extensive user oversight, this system may allow wearers to set specific preferences for data sharing, storage, and deletion. The integration with a decentralized governance system may provide an additional layer of security and transparency, potentially increasing user trust and engagement with the posture improvement program. This approach to data management may represent a novel feature in the field of wearable posture correction devices, addressing growing concerns about data privacy in connected health technologies.

The combination of biomechanical posture support, AI-driven insights, personalized haptic feedback, and user-controlled data management may create a comprehensive real-time health improvement solution that goes beyond the capabilities of existing devices. By enabling broader benefits of posture correction simultaneously, from physical support to behavioral reinforcement and adjustment, and data privacy, the posture support garment device may offer a more holistic and effective approach to improving posture and overall well-being.

### REFERENCE NUMERALS

101 posture support garment device

102 back band portion (dorsal area)

103 axilla region portion

104 shoulder joint

105 sleeve member

106 scapular region portion

107 underarm region

108 forward portion securing end

109 first fastener (left side)

110 second fastener (right side)

111 wearer

501 reinforcement inner layer

502 narrow region of the reinforcement inner layer

503 shoulder joint section

504 shoulder support

505 inner dorsal reinforcement layer

551 support layer adhesive

555 dorsal layer adhesive

561 edge adhesive

562 sleeve adhesive

601 hemmed edge

602 sleeve edge

701 reinforcement inner layer grippy coating

702 deltoid region grip coating

703 dorsal region grippy coating

801 structural support cover

802 reinforcement seams

803 adhesive film

901 boning encasement

902 boning piece

903 structural support element

903A structural support element

903B structural support element

1001 stitching between mirrored halves

1002 sleeve seam

1003 alignment tab

1101 sensor

1102 microcontroller

1103 wireless interface

1104 haptic transducer

1301 user system

1302 front end services

1303 shared public services network

1304 backend system

1305 garment device

1305A garment device

1305B garment device

1305C garment device

1305N garment device

1306 edge device

1306A edge device light node

1306C edge device full node

1306N edge device

1307A shared records

1307B shared records

1307C shared records

1307N shared records

1308 developer APIs

1309 marketplace interface

1310 incentive system interface

1311 exchange engine

1312 rules engine

1313 product services

1314 commerce engine

1315 business engine

1316 analytics engine

1317 business data

1318 analytics data

1319 business data

1401 initializing user-to-system relationship step

1402 use product step

1403 contribute to system step

1404 collect and store usage data step

1405 detect/ascertain contribution step

1406 evaluate data/contribution and terms step

1407 make offer step

1408 acceptance step

1409 settle and exchange credit/stake for data/contribution and terms step

1410 exist without exchange step

1501A heartbeat sensor

1501B moisture sensor

1501C skin temperature sensor

1501D speed sensor

1501N other sensor

1502 monitor step

1503 detection step

1504 determine state step

1505 decision step

1506 active/modulate/deactivate feedback step

1507A running state heartbeat

1507B anxious state heartbeat

1507C temperature state

1507N other state

1508 manual user intervention

A number of implementations have been described. Nevertheless, it will be understood that various modifications may be made without departing from the spirit and scope of the disclosure.

Accordingly, other implementations are within the scope of the following claims.

### CLAUSES

1. A posture support garment device, comprising: a back band portion extending across a wearer's back; an axilla region portion connected to the back band portion and extending under the wearer's axilla region, rising above shoulder joints to provide a supportive lift that pulls shoulders back and downward; a scapular region portion joined to the axilla region portion, forming a cross-back support structure; an underarm region joined to the scapular region portion and extending under the axilla on an opposite side from the shoulder, continuing forward along a torso; and a forward portion originating from the underarm regions and fastening in front.
2. The posture support garment device of clause 1, wherein the axilla region portion includes a sleeve member that encircles the shoulder joints and axilla, extending around the deltoid, biceps, and triceps muscles, and being adaptable in length and shape.
3. The posture support garment device of clause 1, wherein the posture support garment device is constructed from a stretchable material designed to mold to the wearer's body contours and provide adjustable support and tension.
4. The posture support garment device of clause 2, further comprising at least one structural support element integrated within the sleeve member to maintain its shape and fit during movement, thereby mitigating material bunching in the underarm region.
5. The posture support garment device of clause 1, further comprising an inner surface with a reversible, repeatable adhesion substance in at least one area to secure the posture support garment device against the wearer's body and reduce displacement caused by movement.
6. The posture support garment device of clause 1, further comprising a monitoring system configured to: collect wearer data through at least one sensor positioned to monitor the wearer's physiological state; process and store data via functional elements; wirelessly transmit data to an edge device for further computation; analyze the wearer's state using artificial intelligence and machine learning algorithms; and synthesize data based on wearer-specific insights derived from a broader wearer population analysis.
7. The posture support garment device of clause 1, further comprising: at least one haptic device embedded in the posture support garment device, configured to create a responsive, personalized experience for the wearer; a user interface operationally connected to a monitoring system, configured to display information pertinent to the wearer's physiological state and provide controls for the wearer to interact with the monitoring system; and a wireless interface configured to connect to a social element, whereby the wearer can wirelessly receive sensory input from others.
8. The posture support garment device of clause 1, wherein the back band portion extends horizontally between the thoracic and sternoxiphoid plane, the axilla region portion rises above acromial regions, the scapular region portion dynamically distributes tension across the back to assist in muscle engagement and postural adjustment, the underarm region provides a self-stabilizing fit that maintains the posture support garment device's position during wearer movement without external straps, and the forward portion traverses beneath a mammary region from both sides of the wearer's body and fastens securely in front.
9. A system for incentivizing use of a product or device or participation in the system, comprising: at least one sensor configured to detect usage of a product or device; functional elements configured to collect and analyze usage data; a value establishment mechanism configured to establish a unit of value for usage; an exchange engine configured to exchange said unit of value for credit that has spendable or sellable value in the system or stake; and a flexible system configured to allow expansion of the system to motivate other behaviors, and to include other products, devices and contributions to the system.
10. The system of clause 9, wherein the usage is measured based on duration and frequency the posture support garment device is worn by a wearer.
11. The system of clause 9, wherein the credit or stake is a form of digital token used to store value and is redeemable within the system.
12. The system of clause 9, wherein the credit or stake is a form of share or asset that can hold value and can be traded between parties within a user system.
13. The system of clause 9, wherein the system employs a decentralized governance structure, allowing multiple entities to participate without a singular controlling authority.
14. The system of clause 9, wherein the incentive system is integrated with the posture support garment of clause 1 to track wear time, reward participation, and facilitate user engagement based on detected duration and frequency of use.
15. A feedback system comprising: at least one sensor configured to capture a user signal; at least one haptic device configured to provide sensory feedback to a user; functional elements configured to analyze sensor data locally to extrapolate the user's state, or collect and transmit sensor data to a remote system to extrapolate the user's state and receive instructions; wherein the functional elements are configured to execute instructions locally or from a remote engine to actuate the at least one haptic device to assist the user in tuning voluntary actions with elements of the user's autonomic nervous system to boost, dampen, or maintain a particular physiological state.
16. The feedback system of clause 15, wherein the at least one sensor is configured to capture heartbeat data and the at least one haptic device is configured to provide feedback of the heartbeat rhythm to the user such that the user can coordinate physical movement with heart rate.
17. The feedback system of clause 15, wherein the system is integrated with a posture support garment device comprising: a back band portion extending horizontally across a wearer's back between the thoracic and sternoxiphoid plane; an axilla region portion connected to the back band portion on left and right sides of the wearer, extending under the left and right axilla regions of the wearer and rising above left and right shoulder joints and respective acromial regions; a scapular region portion joined to the axilla region portion, forming a cross-back support structure; an underarm region joined to the scapular region portion on left and right sides, extending under the axilla on an opposite side from the shoulder and continuing forward along left and right sides of the wearer's torso; and a forward portion originating from the underarm regions, traversing beneath a mammary region from opposite sides of the wearer's body and fastening in front.
18. A method of incentivizing use of the system of clause 9, the method comprising: providing a device having onboard sensors for tracking and collecting usage data; establishing a connection with the incentive system through an interface on computing device across which data and instructions can be transferred; at predetermined intervals agreed upon by the user through a user interface, collecting sensor data and sharing it with the incentive system; at predetermined intervals, the user's device transmitting data to the incentive system through the interface about an action recognized by the incentive system to have value; evaluating sensor data, and other received data, and applicable terms of use, assessing value and presenting the user an offer in the form of credit or stake understood by the user to have value within the incentive system; settling an exchange agreement if the user accepts the offer either manually or through automation; copying and storing an agreed upon data set and terms into the incentive system; transferring an agreed amount of the offer to the user's account.
19. The method of clause 18, wherein the device is a posture support garment device comprising: at least one sensor configured to detect usage; functional elements configured to collect, transmit, receive and analyze data and instructions locally or from a remote computational engine, the functional elements comprising: data storage, a processor, communication capability with other components or devices, wireless transmission, and a power source; wherein the functional elements are capable of extrapolating and computing product usage from sensor data whereby the date-and-time, frequency, intensity and duration of usage can be determined along with other factors ascertained to be relevant to usage; a value establishment mechanism configured to establish a unit of value for usage or participation whereby a user understands what value to expect in exchange for sharing data and participating in the incentive system; an exchange engine configured to exchange the unit of value for product usage or participation data in a form of credit that has spendable or sellable value in the incentive system; a back band portion extending horizontally across a wearer's back between the thoracic and sternoxiphoid plane; an axilla region portion connected to the back band portion on left and right sides of the wearer, extending under the left and right axilla regions of the wearer and rising above left and right shoulder joints and respective acromial regions; a scapular region portion joined to the axilla region portion, forming a cross-back support structure; an underarm region joined to the scapular region portion on left and right sides, extending under the axilla on an opposite side from the shoulder and continuing forward along left and right sides of the wearer's torso; and a forward portion originating from the underarm regions, traversing beneath a mammary region from opposite sides of the wearer's body and fastening in front.

1. A posture support garment device, comprising:
a back band portion extending across a wearer's back;
an axilla region portion connected to the back band portion and extending under the wearer's axilla region, rising above shoulder joints to provide a supportive lift that pulls shoulders back and downward;
a scapular region portion joined to the axilla region portion, forming a cross-back support structure;
an underarm region joined to the scapular region portion and extending under the axilla on an opposite side from the shoulder, continuing forward along a torso; and
a forward portion originating from the underarm regions and fastening in front.

2. The posture support garment device of claim 1, wherein the axilla region portion includes a sleeve member that encircles the shoulder joints and axilla, extending around the deltoid, biceps, and triceps muscles, and being adaptable in length and shape.

3. The posture support garment device of claim 1, wherein the posture support garment device is constructed from a stretchable material designed to mold to the wearer's body contours and provide adjustable support and tension.

4. The posture support garment device of claim 2, further comprising at least one structural support element integrated within the sleeve member to maintain its shape and fit during movement, thereby mitigating material bunching in the underarm region.

5. The posture support garment device of claim 1, further comprising an inner surface with a reversible, repeatable adhesion substance in at least one area to secure the posture support garment device against the wearer's body and reduce displacement caused by movement.

6. The posture support garment device of claim 1, further comprising a monitoring system configured to:
collect wearer data through at least one sensor positioned to monitor the wearer's physiological state;
process and store data via functional elements;
wirelessly transmit data to an edge device for further computation;
analyze the wearer's state using artificial intelligence and machine learning algorithms; and
synthesize data based on wearer-specific insights derived from a broader wearer population analysis.

7. The posture support garment device of claim 1, further comprising:
at least one haptic device embedded in the posture support garment device, configured to create a responsive, personalized experience for the wearer;
a user interface operationally connected to a monitoring system, configured to display information pertinent to the wearer's physiological state and provide controls for the wearer to interact with the monitoring system; and
a wireless interface configured to connect to a social element, whereby the wearer can wirelessly receive sensory input from others.

8. The posture support garment device of claim 1, wherein the back band portion extends horizontally between the thoracic and sternoxiphoid plane, the axilla region portion rises above acromial regions, the scapular region portion dynamically distributes tension across the back to assist in muscle engagement and postural adjustment, the underarm region provides a self-stabilizing fit that maintains the posture support garment device's position during wearer movement without external straps, and the forward portion traverses beneath a mammary region from both sides of the wearer's body and fastens securely in front.

9. A system for incentivizing use of a product or device or participation in the system, comprising:
at least one sensor configured to detect usage of a product or device;
functional elements configured to collect and analyze usage data;
a value establishment mechanism configured to establish a unit of value for usage;
an exchange engine configured to exchange said unit of value for credit that has spendable or sellable value in the system or stake; and
a flexible system configured to allow expansion of the system to motivate other behaviors, and to include other products, devices and contributions to the system.

10. The system of claim 9, wherein the usage is measured based on duration and frequency the posture support garment device is worn by a wearer.

11. The system of claim 9, wherein the credit or stake is a form of digital token used to store value and is redeemable within system.

12. The system of claim 9, wherein the credit or stake is a form of share or asset that can hold value and can be traded between parties within a user system.

13. The system of claim 9, wherein the system employs a decentralized governance structure, allowing multiple entities to participate without a singular controlling authority.

14. The system of claim 9, wherein the incentive system is integrated with the posture support garment of claim 1 to track wear time, reward participation, and facilitate user engagement based on detected duration and frequency of use.

15. A feedback system comprising:
at least one sensor configured to capture a user signal;
at least one haptic device configured to provide sensory feedback to a user;
functional elements configured to analyze sensor data locally to extrapolate the user's state, or collect and transmit sensor data to a remote system to extrapolate the user's state and receive instructions;
wherein the functional elements are configured to execute instructions locally or from a remote engine to actuate the at least one haptic device to assist the user in tuning voluntary actions with elements of the user's autonomic nervous system to boost, dampen, or maintain a particular physiological state.

16. The feedback system of claim 15, wherein the at least one sensor is configured to capture heartbeat data and the at least one haptic device is configured to provide feedback of the heartbeat rhythm to the user such that the user can coordinate physical movement with heart rate.

17. The feedback system of claim 15, wherein the system is integrated with a posture support garment device comprising:
a back band portion extending horizontally across a wearer's back between the thoracic and sternoxiphoid plane;
an axilla region portion connected to the back band portion on left and right sides of the wearer, extending under the left and right axilla regions of the wearer and rising above left and right shoulder joints and respective acromial regions;
a scapular region portion joined to the axilla region portion, forming a cross-back support structure;
an underarm region joined to the scapular region portion on left and right sides, extending under the axilla on an opposite side from the shoulder and continuing forward along left and right sides of the wearer's torso; and
a forward portion originating from the underarm regions, traversing beneath a mammary region from opposite sides of the wearer's body and fastening in front.

18. A method of incentivizing use of the system of claim 9, the method comprising:
providing a device having onboard sensors for tracking and collecting usage data;
establishing a connection with the incentive system through an interface on computing device across which data and instructions can be transferred;
at predetermined intervals agreed upon by the user through a user interface, collecting sensor data and sharing it with the incentive system;
at predetermined intervals, the user's device transmitting data to the incentive system through the interface about an action recognized by the incentive system to have value;
evaluating sensor data, and other received data, and applicable terms of use, assessing value and presenting the user an offer in the form of credit or stake understood by the user to have value within the incentive system;
settling an exchange agreement if the user accepts the offer either manually or through automation;
copying and storing an agreed upon data set and terms into the incentive system;
transferring an agreed amount of the offer to the user's account.

19. The method of claim 18, wherein the device is a posture support garment device comprising:
at least one sensor configured to detect usage;
functional elements configured to collect, transmit, receive and analyze data and instructions locally or from a remote computational engine, the functional elements comprising: data storage, a processor, communication capability with other components or devices, wireless transmission, and a power source;
wherein the functional elements are capable of extrapolating and computing product usage from sensor data whereby the date-and-time, frequency, intensity and duration of usage can be determined along with other factors ascertained to be relevant to usage;
a value establishment mechanism configured to establish a unit of value for usage or participation whereby a user understands what value to expect in exchange for sharing data and participating in the incentive system;
an exchange engine configured to exchange the unit of value for product usage or participation data in a form of credit that has spendable or sellable value in the incentive system;
a back band portion extending horizontally across a wearer's back between the thoracic and sternoxiphoid plane;
an axilla region portion connected to the back band portion on left and right sides of the wearer, extending under the left and right axilla regions of the wearer and rising above left and right shoulder joints and respective acromial regions;
a scapular region portion joined to the axilla region portion, forming a cross-back support structure;
an underarm region joined to the scapular region portion on left and right sides, extending under the axilla on an opposite side from the shoulder and continuing forward along left and right sides of the wearer's torso; and
a forward portion originating from the underarm regions, traversing beneath a mammary region from opposite sides of the wearer's body and fastening in front.
